(19) United States
(12) Patent Application Publication
(10) Pub. No.: US 2021/0284944 A1
(43) Pub. Date: Sep. 16, 2021

(54) HIGH-THROUGHPUT MULTI-ORGAN PERFUSION MODELS

(71) Applicant: Novartis AG, Basel (CH)

(72) Inventors: Taraka Sai Pavan GRANDHI, San Diego, CA (US); Scott HAMMACK, San Diego, CA (US); Shane HORMAN, Encinitas, CA (US); Cody SCANDORE, San Francisco, CA (US); Gary SEEBOLD, Santee, CA (US)

(21) Appl. No.: 17/332,166

(22) Filed: May 27, 2021

Related U.S. Application Data
(63) Continuation of application No. PCT/IB2019/001295, filed on Nov. 27, 2019.
(60) Provisional application No. 62/772,935, filed on Nov. 29, 2018.

(51) Int. Cl. C12M 1/42; C12M 1/32; C12M 1/00; C12M 1/12

(52) U.S. Cl. CPC C12M 35/04; C12M 23/12; C12M 25/04; C12M 37/04; C12M 23/40

(57) ABSTRACT

The invention provides a fluidic device that comprises at least two separate testing units, each of which is adapted to expose living cells to a moving fluid. The fluidic devices are useful for testing cell types such as kidney cells that are sensitive to shear stress, and can be configured for high-throughput testing. The fluidic device is adapted to receive a multi-well cell culture plate to which the living cells can be adhered or affixed. In some embodiments, flow channels in the fluidic device are positioned to expose the living cells to moving fluid, and the flow wells are adapted to provide substantially uniform shear stress across the area where the living cells are exposed to the moving fluid.

### CROSS-REFERENCES TO RELATED APPLICATIONS

This application is a continuation of International Application PCT/IB2019/001295, filed Nov. 27, 2019, which claims the benefit of U.S. Application No. 62/772,935, filed Nov. 29, 2018, each of which are incorporated by reference herein.

### TECHNICAL FIELD

This invention relates to a device for growing cells that recapitulate shear-sensitive phenotypes found in living tissues and organs, and methods to test how such cells are affected by or interact with exogenous substances and influences.

### BACKGROUND ART

The human body is an interconnected collection of different organs and tissues that communicate with one another. Because of the interactions between different organs, many disease states such as cancer metastases, acute kidney injury and gut-brain disorders and in-vitro drug pharmacodynamics and pharmacokinetics are not well understood by examining one organ or tissue. Biological microfluidic model systems of kidney, liver, heart and lung microenvironments have improved the understanding of the individual organs, and increasingly those models are being connected to explore crucial crosstalk between different organ systems to support research such as drug discovery, organ development, and toxicology studies. These model systems can provide valuable insights for medical and pharmaceutical research such as studying toxicity, metabolism, and pharmacokinetics of drugs or potential new drugs.

Many cells and tissues are exquisitely sensitive to seemingly subtle physical forces that occur in the body: such forces can significantly change the cell membrane and its properties in tissues like kidney, heart, and liver in vivo. For example, fluid shear created by continuous movement of glomerular filtrate in the kidney proximal tubule environment causes functional maturation and activation of the tubular epithelial cells, allowing increased reabsorption relative to cells developing in the absence of shear stress: traditional high-throughput model systems do not reproduce this effect. Thus, while conventional static cell cultures are certainly useful for exploring many aspects of cell physiology and function, they do not provide accurate information about important aspects of cells that are influenced by physical forces such as fluid flow or motion. Specialized systems and devices have been developed to simulate these forces so that organ-specific conditions that are heavily influenced by those forces can be investigated.

In-vitro model organs often utilize microfluidic systems, or can be adapted to a microfluidic platform to increase throughput of testing. Microfluidic systems are fluid-handling systems that have fluid transfer components of sub-millimeter size (ca. 100-1000 μm). Recent efforts and advances in microfluidics have allowed the miniaturization of biological systems into devices comparable in size to a US penny. Some of the existing devices are designed to expose cells to shear stress, but are principally designed to enable visual observation of cell adhesion dynamics, and are not suitable for high-throughput screening or simulation of interactions between multiple cell types (U.S. Pat. No. 9,599,604).

Biological phenotypes that depend on fluid shear for their expression can be reproduced by some specialized microfluidic devices. For example, renal proximal tubule epithelial cells grown on filter membrane surfaces and exposed to fluid shear in a microfluidic device, manifest pathologies similar to those exhibited in vivo by proximal tubules. Endothelial cells grown on microfluidic chambers and exposed to appropriate fluid shear mature and express physiological markers similar to cells in an in-vivo environment. Recently, as mentioned before, separate microfluidic devices representing cells of multiple organs or tissues have also been integrated into one interconnected multi-organ model system for drug efficacy and toxicity modeling in a realistic multi-organ environment. Recent work by Skardal et al., showed integration of heart, liver and lung microfluidic modules into a connected microfluidic system. Multi-tissue interactions in an integrated three-tissue organ-on-a-chip platform, Scientific Reports, 7(1), 8837 (2017). This multi-organ system was used to study the toxicity of bleomycin under increasingly relevant in vivo model conditions, and bleomycin was shown to induce lung inflammatory factor-driven cardiotoxicity in this system.

While microfluidic systems offer numerous advantages for tissue modeling such as providing relevant fluidic forces, physiologically relevant cells to surrounding fluid ratio, known ones do not readily adapt for use in high throughput drug discovery efforts. Jang, et al., Integrative Biology, 5(9), 1119-29 (2013). Doriot, et al., Coronary artery disease, 11(6), 495-501 (2000). Chang, et al., JCI Insight, 2(22), 2017. Bauer, et al., Scientific Reports 7(1), 14620 (2017). Most of the existing embodiments of single-tissue microfluidic devices require at least three components per unit: an inlet pipe, an outlet pipe and a microfluidic biological space in between the two (WO2017/176357). Since each test unit requires two ‘pipes’, miniaturizing and multiplying this design into dense plate formats that mimic physiological conditions such as shear stress poses a number of challenges. For example, a 96 well microfluidic system would require management of at least 192 pipe embodiments when separation from well to well is required. While it is easy to create programmable interconnected arrays in microfluidic systems, the requirement of extensive pipe systems to channel fluids hamper their translation into high-throughput scale suitable for use with conventional HTS robotics systems and commercial 96-well and higher-density plate formats.

There is a significant need for microfluidics systems that accurately simulate individual organs and combinations of organs, and are capable of being integrated into high throughput, automated format for drug discovery and development. The present invention provides such devices and methods that accurately simulate in vivo conditions for cell, tissue and organ types whose development is sensitive to shear forces, such as microtubule structures in the kidney.

### SUMMARY OF THE INVENTION

In one aspect, the present invention provides a fluidic device that exposes living cells to fluid shear stress in a consistent and reproducible manner. The device can be adapted for use in the testing of living cells exposed to shear stress as a model organ for testing how the cells and the organ behave, and of their interaction with exogenous effectors and/or other model organs. Moreover, the device can contain many separate individual units, each having its own separate cell population and fluid environment, where each unit provides the same amount of shear stress or shear force on the living cells, and the device can include many such units, such as 96 units or 384 units or 1536 units, to conform to array sizes commonly used in high-throughput testing systems and protocols. Thus the device can be used for high-throughput testing in automated systems such as those conventionally used in various research programs and assay systems for testing of biochemical and biological model systems. This device is a significant advance toward creating model systems that accurately represent interactions among key components of the human body that advance pharmaceutical and medical research, and can help reduce the use of live animals in medical and pharmaceutical testing.

In one aspect, the invention provides a fluidic device comprising:
a manifold body comprising a substantially flat horizontal top surface, two or more separated flow channels, each of said flow channels having a horizontal section with a horizontal width, wherein each flow channel is connected to an inlet channel and an outlet channel, wherein the inlet channel and the outlet channel extend from the flow channel to the exterior of the manifold body;
the manifold body further comprising two or more holes extending downward from the top surface into the manifold body, wherein each of said holes intersects the horizontal section of one flow channel,
wherein each flow channel is configured so that fluid moving from the inlet channel through the horizontal section of the flow channel toward the outlet channel must pass through the portion of the flow channel that intersects at least one of the holes extending downward from the top surface of the manifold body. Note that naming of the inlet and outlet channels is somewhat arbitrary, as the roles of the two channels depend upon how the fluidic device is used. In some embodiments, the manifold body is constructed of two pieces. Optionally, the width of each hole where it intersects the flow channel is greater than the horizontal width of the flow channel at the point where the hole and flow channel intersect by a small amount. Optionally, additional layers can be added to the bottom of the manifold body.

In another aspect, the invention provides the fluidic device described above in combination with a multi-well cell culture plate adapted to be fitted to the fluidic device. The multi-well cell culture plate is adapted to provide a support or substrate suitable for growing living cells, either inside the wells of the cell culture plate or adhered to the bottoms of the wells where the cells can be exposed to liquid flowing in the flow channels of the fluidic device of the invention. In some embodiments, the cell culture plate is configured with 96 wells or more, such as 384 wells or 1536 wells. The multi-well cell culture plate can be selected from ones commercially available, and the fluidic device can be sized and configured to match such commercial cell culture plates.

In another aspect, the invention provides a system comprising the fluidic device described herein and a mechanism to cause fluid flow within the flow channels inside the manifold body of the fluidic device. In typical embodiments, the mechanism is capable of causing fluid to flow within the horizontal section of the flow channel of the fluidic device. In some of these systems, the mechanism to move fluid within the flow channel is selected from a pressure pump, a suction pump, flexible membrane disposed across an opening of the inlet channel or outlet channel of the fluidic device that can be deflected to move fluid, a bulb or balloon configured to apply suction or pressure to the fluid via the inlet channel or outlet channel of the fluidic device, and a mechanism to physically move the fluidic device in a reciprocating manner.

In another aspect the invention provides methods to use the fluidic devices and systems described herein to determine actions and/or reactions of living cells, especially living cells exposed to shear stress created by fluid flow in the flow channels of the fluidic devices of the invention. In some embodiments, the methods provide a model organ for various tests, either in isolation or in interactive combination with other model organs.

In another aspect, the invention provides a device to expose living cells to fluid shear stress, wherein the device comprises:
a plurality of wells having generally vertical walls and a generally horizontal floor, wherein at least a portion of the floor is a permeable membrane;
at least one flow channel positioned below the wells so that the permeable membrane portion of the floor of each well separates the well from one of the at least one flow channels; and
an inlet that connects the flow channel to the exterior of the device, and an outlet that connects the flow channel to the exterior of the device, wherein a fluid path leading from the inlet, through the flow channel to the outlet passes beneath the permeable membrane portion of the floor of at least one well.
Preferably, the plurality of wells are connected together by a generally horizontal plate in the form of a multi-well cell culture plate, and the multi-well cell culture plate can be separated from the remainder of the device.

The invention provides a method to use the device to expose living cells to fluid shear, which comprises placing cells on the permeable membrane of the device, and contacting the living cells with fluid moving within a flow channel of the device.

The following examples and detailed description highlight other aspects and embodiments of the invention. Other objects, advantages and features of the present invention will be apparent from the following specification taken in conjunction with the accompanying drawings.

### BRIEF DESCRIPTION OF THE DRAWINGS

FIG. 1 is a side view of a cross-section of a testing unit of the fluidic device.

FIG. 2A is a side view of a single testing unit of the device.

FIG. 2B is a side view of a single testing unit with an added O-ring (11b).

FIG. 3 depicts a top view of a 96-well cell culture plate for use with certain embodiments of the fluidic device.

FIG. 4A and FIG. 4B show the upper portion and lower portion, respectively, of a two-piece manifold body.

FIG. 4C shows the upper surface (top panel) and lower surface (lower panel) of the upper portion of a two-piece manifold body, including

FIG. 4D shows the upper surface (top panel) and lower surface (lower panel) of the lower portion of a two-piece manifold body, including grooves (13b) for holding O-rings around the openings of the inlet channels (13a).

FIG. 5A is an exploded view of an embodiment of the fluidic device, shown with a cell culture plate (9) above the upper portion (14) of a two-piece manifold body; a layer of O-rings (11a) between the upper (14) and lower (15) portions of the manifold body; a flexible membrane (17) to be applied (optionally glued) to the bottom of the lower portion (15) of the manifold body; and a support plate (18) to hold the flexible membrane in place, optionally using screws (20) to attach the support plate to the lower portion of the manifold body.

FIG. 5B is an exploded view of an embodiment of the fluidic device, shown with a cell culture plate (9) above the upper portion (14) of a two-piece manifold body; a layer of O-rings (11a) between the upper and lower portions of the manifold body; a second layer of O-rings (11b) to go between the lower surface of the manifold body and the flexible membrane (17), which can be applied (optionally glued) to the bottom of the lower portion (15) of the manifold body; and a support plate (18) to hold the flexible membrane in place, optionally using screws (20) to attach the support plate to the lower portion of the manifold body using extensions (ears) (19).

FIG. 6 illustrates how the opening of an outlet channel that opens to the bottom of the manifold body can be enlarged, or flared, to increase the area of the flexible membrane that can flex either up or down in order to increase the extent of fluid motion induced by membrane flexion, without increasing the applied force on the membrane.

FIG. 7A shows the results obtained from fluid dynamic simulation for two separate trials. The boxed segment indicates the measurements in Trial 1 within a range of 5% standard deviation. The boxed segment for Trial 2 (second panel) shows measurements with 4% standard deviation.

FIG. 7B and FIG. 7C depict a flow channel shape selected based on fluid dynamic simulations for a flow channel that intersects with a single well (7B) and a flow channel that intersects three wells (7C).

FIG. 8 illustrates a method of using a single testing unit to assess interactions between two different types of cells, liver cells in the left well and kidney cells in the right well, thus simulating a liver organ interacting with a kidney organ.

FIG. 9 illustrates how the fluidic device can be used to simulate interactions between two different organs having different shear stress environments, simulated by three wells on the right side carrying kidney cells interacting with one well on the left having heart cells. Below the image of the pattern of wells is an illustration of a Transwell® plate configured to provide 8 repetitions of the 4-well combination.

FIG. 10A represents the three test units for the experiment in Example 5, where each test unit contains two connected wells, one of which has medium (or medium and nuclear stain) while the other well contains HCT116 cells.

FIG. 10B shows images of cells in the collagen-free experiment at the beginning of the experiment, and at 3 hours and 24 hours of operation according to Example 5. The front panel is live/dead staining to confirm the cultured cells were live at the start of the experiment; the other two panels show migration of stain in the middle test unit with no contamination of wells adjacent to the test unit at 3 hours, and increased staining of cells in the middle test unit with virtually no migration of stain to the adjacent test units.

FIG. 10C shows results of the collagen-containing experiments in Example 5, showing that collagen plug in the test unit greatly slowed migration of the nuclear stain, but that migration to the connected well still occurred at 24 hours, again with little or no migration to non-connected adjacent wells.

FIG. 11A represents the three test units for the experiment in Example 6, where each test unit contains two connected wells, one of which has medium with indicated amounts of staurosporine (0 μM, 25 μM and 50 μM), while the other well contains HCT116 cells.

FIG. 11B shows images of cells in the cell containing wells of experiment in Example 6, after 24 hours of operation according to Example 6. The top panel shows dose-dependent cell death in the HCT116 cells. The second panel shows that collagen plug largely suppressed migration of staurosporine to the cell-containing wells, as evidenced by little cell death. The bar graph quantifies the extent of cell death for the collagen-free and collagen-containing test units.

FIG. 12A depicts the two wells of a test unit according to Example 7, where the first well has hepatocytes inside, and prodrug of 5-fluorouracil (5-FU) is added, while the second well contains HCT116 cells expected to be killed by 5-FU if it is produced by the hepatocytes from the prodrug.

FIG. 12B illustrates the operation of a test unit according to Example 7, where operation of the device is expected to allow 5-FU formed in the hepatocyte-containing wells to migrate to the connected well, containing HCT116 cells.

FIG. 12C shows images of the hepatocytes (first panel) and dose-dependent death of HCT116 cells in the connected wells after operation of the device for 24 hours (two iterations of the experiment are shown, using prodrug concentrations of 0, 100, 200, 300 and 400 uM). Again, the images of the HCT116 cells in each well are shown as four quadrants.

FIG. 13A shows that human Proximal tubule epithelial cells (PTECs) maintained on a Transwell® membrane under static conditions in the device for 48 hours (image 2) exhibit similar viability as PTECs on a control well membrane (image 1).

FIG. 13B shows that PTECs maintained on a Transwell® membrane for 12 hours under fluid flow conditions that expose the cells to 0.1-0.2 dynes/cm² shear (image 2, fluid shear conditions in the device for 12 hours) exhibit similar viability as PTECs on a control membrane well (image 1).

### DETAILED DESCRIPTION OF THE INVENTION

Unless defined otherwise, or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by

### REFERENCE

### Definitions

As used herein, the singular forms “a”, “an”, and “the” include plural references unless indicated otherwise. For example, “a” dimer includes one or more dimers.

As used herein, the term “microfluidic device” generally refers to a device through which materials, particularly fluid borne materials, such as liquids, can be transported, in some embodiments on a micro-scale, and in some embodiments on a nanoscale. Thus, the microfluidic devices described herein can comprise microscale features, nanoscale features, and combinations thereof. Because the devices described herein are primarily useful for growing cells and experimenting on living cells, the microfluidic features are typically optimized to handle aqueous solutions.

Accordingly, an exemplary microfluidic device typically comprises structural or functional features dimensioned on the order of a millimeter-scale or less, which are capable of manipulating a fluid, typically aqueous fluid, at a flow rate on the order of a μL/min or less. Typically, such features include, but are not limited to channels, fluid reservoirs, reaction chambers, mixing chambers, and separation regions. In some examples, the channels include at least one cross-sectional dimension that is in a range of from about 0.1 μm to about 500 μm. The use of dimensions on this order allows the incorporation of a greater number of channels in a smaller area, and utilizes smaller volumes of fluids.

A microfluidic device can exist alone or can be a part of a combination or a system which, for example and without limitation, can include: a multi-well cell culture plate, a pump for introducing fluids, e.g., samples, reagents, buffers, nutrients, and the like, into the system and/or moving fluids within the device or through the system; detection equipment or systems; data storage systems; and control systems for controlling fluid movement and/or direction within the device, monitoring and controlling environmental conditions to which fluids in the device are subjected, e.g., temperature, flow rate, electric voltage or current, and the like.

As used herein, the terms “channel” refers to a recess or cavity formed in a material to contain fluid or transmit fluid. A channel can be formed by imparting a pattern from a patterned substrate into a material or by any suitable material removing technique. In the present invention, channel size means the cross-sectional area of a fluidic or microfluidic channel.

It is understood that aspects and embodiments of the invention described herein, while described as comprising specified features, also include embodiments “consisting of” and/or “consisting essentially of” the specified features and limitations.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “about” as used herein indicates that a reasonable amount of variation can occur without departing from the structure, function, or effect being described. Unless otherwise specified, ‘about’ a numeric value refers to a range around the specified value of +/−10% of the numeric value, and preferably +/−5% of the numeric value.

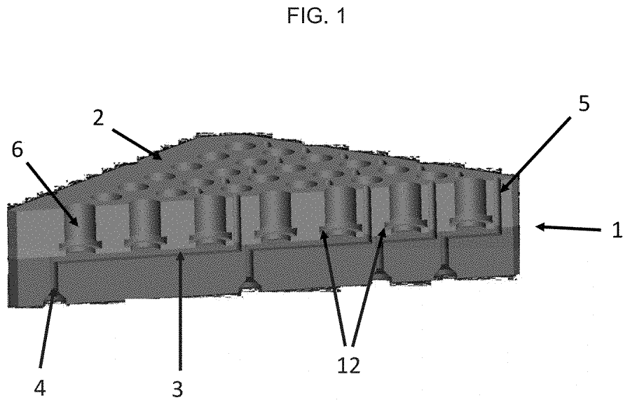

The invention provides a device for exposing a cell or matrix of cells to shear force, which is useful for growing and exploring types of cells and tissues under conditions closely corresponding to their in vivo state. This is especially valuable for analyzing or testing cell types that are known to function in an environment where they are exposed to shear fluid flow forces, and for cell types that exhibit shear-dependent phenotypes or functions. The device comprises a manifold body that is configured to accept the wells of a multi-well cell culture plate fitted into a plurality of holes that extend downward from the top surface of the manifold body. FIG. 1 provides a cross-sectional view of an embodiment of the manifold body (1) of the invention, configured for use with the Corning Transwell® plates described herein, showing the holes (6) that extend downward from the top surface (2), which are positioned and configured to accept the wells of a multi-well cell culture plate. FIG. 1 also shows how horizontal sections of flow channels (3) intersect the bottoms of those holes, and where the inlet (4) and outlet (5) channels that enable fluid to flow through the flow channel connect the flow channel to the exterior.

The device is very flexible in application: it can be configured to represent one tissue or cell type alone, or multiple types of cells for use as a model of interactions between organs or tissues. The device can be autoclaved for sterilization when suitable materials are used, and such embodiments are especially useful. It can be centrifuged if needed to promote complete loading of materials into wells, channels or chambers, and to hasten removal of bubbles from the fluids the device is designed to receive, contain or transport.

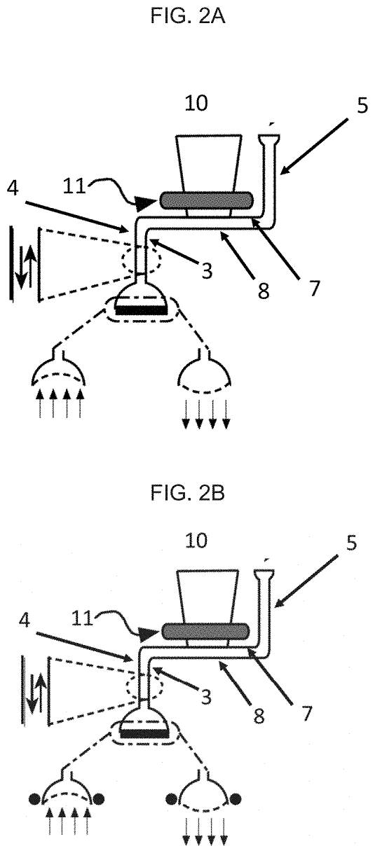

In addition, the device of the invention can be constructed with many individual testing units combined into a single manifold body that can be sized for use in high-throughput screening methods and can be compatible with existing commercial multi-well cell culture plates such as 4-well and 96-well Transwell® plates made by Corning. A testing unit comprises a hole in the manifold body to accept a well of a multi-well cell culture plate, and a horizontal section of a flow channel intersecting the hole, where the horizontal section of the flow channel is connected to an inlet channel and an outlet channel to enable fluid flow through the horizontal section of the flow channel. FIG. 2 illustrates a testing unit, showing the well and an O-ring around (11a) it to form a seal with the manifold body, and a horizontal section of flow channel, and also indicates one mechanism to drive fluid motion within the horizontal section of the flow channel. The mechanism in this embodiment is a flexible membrane across the opening of the inlet channel, which can flex up and/or down, causing fluid within the flow channel to move, as indicated by the up-and-down arrows in the inset of the inlet channel (4). FIG. 2B illustrates where a second O-ring (11b) is optionally used to form a seal around the inlet where it opens on the lower surface of the manifold body. Optional O-ring 11b can be used to improve the seal around the inlet where it sits on a flexible membrane that can be used to drive fluid motion as illustrated in FIGS. 2A and 2B.

In some embodiments, the manifold body of the device of the invention is sized and configured for use with a 96-well Corning Transwell® plate, having 96 wells arrayed as further described herein. Use of the Corning Transwell® plate allows cells to be applied to the underside of the wells, permitting each well to have its own, isolated area of live cells, so that cells of each well can be matured under suitable conditions, and the wells can be matured differently if needed. Thus cells of different types can be connected in series if desired, since fluid in one flow channel can be in contact with two or more wells.

The following enumerated embodiments describe some aspects and embodiments of the invention.
1. A fluidic device comprising:

a manifold body comprising a substantially flat horizontal top surface, two or more separated flow channels, each of said flow channels having a horizontal section with a horizontal width, wherein each flow channel is connected to an inlet channel and an outlet channel, wherein the inlet channel and the outlet channel extend from the flow channel to the exterior of the manifold body;

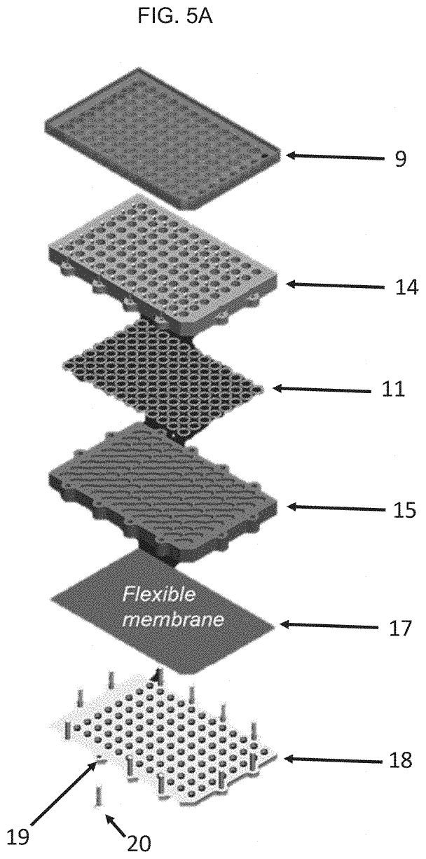

the manifold body further comprising two or more holes extending downward from the top surface into the manifold body, wherein each of said holes intersects the horizontal section of one flow channel,

wherein each flow channel is configured so that fluid moving from the inlet channel through the horizontal section of the flow channel toward the outlet channel must pass through the portion of the flow channel that intersects at least one of the holes extending downward from the top surface of the manifold body; and wherein, optionally, the width of each hole where it intersects the flow channel is greater than the horizontal width of the flow channel at the point where the hole and flow channel intersect by a small amount.
In some embodiments, the manifold body is a rectangular cuboid, wherein each face is generally rectangular in shape, and the manifold body has a length (L), a width (W) and a height (H).
Typically, the flow channel is a generally horizontal opening or void within the manifold body for holding fluid, where fluid can flow from one end of the flow channel to the other end by entering and exiting the flow channel through the inlet channel and outlet channel. The horizontal section of the flow channel is generally rectangular in its vertical cross-section across its width, thus having a ceiling and a floor that are horizontal, and walls that are vertical. The flow channel extends across and intersects with at least one of the holes that extend downward from the top surface of the manifold body.
Typically, the inlet channel is a generally cylindrical hole (though it can be of any convenient shape) that connects with the horizontal section of the flow channel at or near one end of the flow channel. It connects to the flow channel, and extends to open on an external surface of the manifold body to permit fluid to be introduced into the flow channel.
Typically, the outlet channel is a generally cylindrical hole (though it can be of any convenient shape) that connects with the horizontal section of the flow channel at or near the opposite end of the flow channel from where the inlet channel connects with the horizontal section of the flow channel. It connects to the flow channel, and extends to open on an external surface of the manifold body to permit fluid to escape from the flow channel. Note, too, that the inlet and outlet channel labels are somewhat arbitrary, since each of the two channels can be used as inlet or outlet depending on how the fluidic device is operated.
2. The fluidic device of embodiment 1, which comprises four or more holes that extend downward from the top surface into the manifold body. In some embodiments, the manifold body comprises 96 holes, or 384 holes, or 1536 holes extending downward from the top surface into the manifold body, serving to accept wells of a multiwell cell culture plate; optionally the holes are arrayed in the same spacing and format as conventional multi-well cell culture plates.
3. The fluidic device of embodiment 1 or embodiment 2, wherein the holes that extend downward from the top surface into the manifold body are circular in horizontal cross section. These holes are sized, configured and positioned to accept the wells that extend downward from the plate of a multi-well cell culture plate.
4. The fluidic device of any one of the preceding embodiments, wherein the holes that extend downward from the top surface into the manifold body are sized and positioned to receive the wells of a multi-well cell culture plate.
5. The fluidic device of any one of the preceding embodiments, wherein each of the two or more holes that extend downward from the top surface into the manifold body ends where the hole intersects a flow channel within the manifold body. The holes thus open into the ceiling of a flow channel, and permit the fluid in the flow channel to be in contact with the bottom of a well of a multi-well cell culture plate when such cell culture plate is fitted to the top of the manifold body so that the wells extend into these holes.
6. The fluidic device of embodiment 5, which further comprises a sealing surface associated with each of said holes, wherein said sealing surface is configured to form a liquid-tight seal between a surface of the manifold body and an outer surface of a well of a multi-well cell culture plate when the multi-well cell culture plate is fitted to the top surface of the manifold body with the wells of the multi-well cell culture plate inserted into the holes that extend downward from the top surface into the manifold body.
7. The fluidic device of embodiment 6, wherein the sealing surface is a gasket, which is optionally configured to be interposed between the manifold body and a multi-well cell culture plate when a multi-well cell culture plate is fitted to the manifold body.
8. The fluidic device of embodiment 6 or embodiment 7, wherein the sealing surface is positioned on the inside of each of the holes that extend downward from the top surface into the manifold body.
9. The fluidic device of embodiment 8, wherein the sealing surface is a sleeve or an O-ring positioned inside each of the holes that extend downward from the top surface into the manifold body. In some of these embodiments, the sealing surface is an O-ring. Optionally, the holes extending downward from the top surface of the manifold body have a groove along the perimeter inside each of said holes, and the groove is positioned and sized to hold an O-ring in place so the O-ring can provide a seal between the surfaces of the hole and the outer surface of a well of the multi-well cell culture plate when the well is inserted into the hole. FIG. 1 depicts an embodiment of the manifold body having a groove (12) along the perimeter inside each of the holes that receives a well of a multi-well cell culture plate.
10. The fluidic device of any of the preceding embodiments, wherein the inlet channel for each flow channel extends downward from one end of the flow channel to open at the bottom surface of the manifold body. Optionally, the manifold body comprises a groove around at least one of the outlet channel openings in the bottom surface of the manifold body, wherein each said groove is configured to hold an O-ring that is positioned to form a seal between the manifold body and the surface beneath the manifold body when the fluidic device is assembled, e.g., the O-ring may form a seal with the flexible membrane beneath the manifold body in the embodiment of FIG. 5B.
11. The fluidic device of any of the preceding embodiments, wherein the outlet channel for each flow channel extends upward from one end of the flow channel to open at the top surface of the manifold body. The inlet channel and outlet channel connect with opposite ends of the flow channel.
12. The fluidic device of any of the preceding embodiments, wherein the holes that extend downward from the top surface into the manifold body are uniform in size and each hole is shaped as a right cylinder having a vertical central axis.
13. The fluidic device of embodiment 12, wherein the vertical central axis of each hole that extends downward into the manifold body is aligned with the midline of the horizontal section of the flow channel the hole intersects.
14. The fluidic device of embodiment 13, wherein the diameter of each of the holes is greater than or equal to the horizontal width of the flow channel where the hole intersects the flow channel so that the outer surface of the wells that are to be inserted into the holes when a multi-well cell culture plate is fitted to the top surface of the manifold body extends horizontally beyond the flow channel, and the bottom edge of the wells extends slightly over the top of the vertical walls that define the flow channel.
15. The fluidic device of embodiment 14, wherein the length of the horizontal section of the flow channel between the inlet channel and the outlet channel is greater than the diameter of a hole that intersects the flow channel. Typically, the inlet channel and the outlet channel are vertical tubes and often are smaller in diameter than the hole that extends downward from the top surface of the manifold body; inlet channels and outlet channels can extend either upward or downward from the flow channel they connect to, and they extend to and open at the surface of the manifold body, permitting fluid to enter and leave the flow channel. The length of the flow channel is typically at least twice the diameter of the hole extending downward from the top surface of the manifold body, and optionally at least three times the diameter of said hole.
16. The fluidic device of any of the preceding embodiments, wherein at least two of the holes that extend downward into the manifold body intersect with a single flow channel. In these embodiments, one end of the flow channel connects to an inlet channel, the flow channel extends from the end connected to the inlet channel across two adjacent holes that extend downward into the manifold body, and extends past the holes to end at the point where the flow channel connects with the outlet channel.
17. The fluidic device of any of the preceding embodiments, wherein each flow channel connects to at least one outlet channel or one inlet channel that extends upward to form an opening at the top surface of the manifold body. Optionally, the opening in the top surface is flared.
18. The fluidic device of any one of the preceding embodiments, wherein the inlet channel is a cylindrical tube that extends downward from the flow channel, and the diameter of the inlet channel increases as the inlet channel approaches the point where it opens on the bottom surface of the manifold body, i.e., the opening of the inlet channel is flared.
19. The fluidic device of any of the preceding embodiments, wherein the outlet channel of each flow channel extends upward to an opening in the top surface of the manifold body. Optionally, the outlet is a generally vertical tube, the upper end of which is an opening in the top surface of the manifold body.
20. The fluidic device of any one of the preceding embodiments, wherein the holes that extend downward from the top surface into the manifold body have a circular horizontal cross-section and are perpendicular to the top surface of the manifold body.
21. The fluidic device of any one of the preceding embodiments, having a multi-well cell culture plate fitted to the top surface of the manifold body so that each well of the multi-well cell culture plate is inserted into one of the holes in the top surface of the manifold body. In these embodiments, the fluidic device further comprises a multi-well cell culture plate assembled with the manifold body. In a preferred embodiment, the multi-well cell culture plate is a 96 well, 384 well, or 1536 well plate, and the bottom of each well comprises a permeable membrane that is positioned to be in contact with fluid in the horizontal section of a flow channel within the manifold body.
22. The fluidic device of embodiment 21, wherein each flow channel is configured to provide substantially uniform shear stress across the bottom surface of a well of the multi-well cell culture plate that is fitted to the top surface of the manifold body.
23. The fluidic device of any one of the preceding embodiments, wherein the manifold body is a two-piece manifold body, which comprises an upper portion (or plate) and a lower portion (or plate), wherein the upper portion has a substantially flat lower surface that is parallel to the horizontal top surface of the manifold body; and the lower portion has a substantially flat upper surface having canals formed therein, wherein the lower surface of the upper portion forms a ceiling over the canals when the upper portion and lower portion are assembled by being pressed together, and optionally having a gasket sandwiched between the upper portion and lower portion to provide a fluid-tight seal between the two portions, and optionally having the upper portion and lower portion glued together, to form an assembled manifold body, so that the canals in the upper surface of the lower portion taken together with the lower surface of the upper portion form the flow channels in the assembled manifold body.
24. The fluidic device of embodiment 23, wherein the inlet for each flow channel extends downward through the lower portion.
25. The fluidic device of embodiment 23 or embodiment 24, wherein the outlet for each flow channel extends upward through the upper portion.
26. The fluidic device of any of the preceding embodiments, wherein the manifold body comprises 96 holes extending downward from the top surface of the manifold body into the manifold body, where said holes are arranged in an 8×12 array. Optionally the spacing of the holes is selected to correspond to the well placement of a 96-well cell culture plate, such as a Corning Transwell® plate
27. The fluidic device of any one of embodiments 1-26, further comprising a flexible membrane applied to a surface of the manifold body covering the openings of the inlet channels, wherein the flexible membrane forms a fluid-tight seal with the manifold body across each of the openings of the inlet channels. Typically, the flexible membrane extends across openings in the bottom of the manifold body. The flexible membrane is optionally made of PDMS or TPU. In a certain embodiment, the flexible membrane is glued/adhered/chemically bonded to the manifold body, often to the lower surface of the manifold body. In some of these embodiments, the flexible membrane is specially adapted for a particular manifold body, and the thickness can vary. In some such embodiments, the flexible membrane has areas that are thinner where the flexible membrane extends across openings in the bottom of the manifold body, and thicker between these openings to ensure an effective seal between the bottom of the manifold body and the platform on which the manifold can be mounted when in operation, such as when used in the method of embodiment 40.
28. The fluidic device of embodiment 27, further comprising means to apply pressure to the portion of the flexible membrane that covers an opening of an inlet channel to cause the flexible membrane to deflect. In these embodiments, a surface of the flexible membrane is in contact with fluid in the inlet channel.
29. The fluidic device of embodiment 28, wherein the means to apply pressure to a portion of the flexible membrane covering an opening of an inlet channel is configured to be able to apply gas pressure or vacuum or both to the portion of the flexible membrane that covers an opening of an inlet channel, and said pressure is typically applied to the side of the flexible membrane that is not in contact with the surface of the manifold body. In these embodiments, flexing of the flexible membrane where it covers an opening of an inlet channel drives causes fluid in the inlet channel to move; in particular rhythmic flexing of the flexible membrane where it covers an opening of an inlet channel, which moves the flexible membrane up and down, drives corresponding movement of the fluid in the inlet channel.
30. The fluidic device of embodiment 29, wherein the means to apply pressure comprises a source of pressurized gas or a vacuum source, and is configured to controllably apply positive or negative gas pressure to the flexible membrane, wherein the amount of pressure is sufficient to cause the flexible membrane to deflect.
31. The fluidic device of any one of embodiments 27-30, which further comprises a support plate that holds the flexible membrane securely against an outside surface of the manifold body, and said support plate has an opening aligned with each of the inlet channel openings that is covered by the flexible membrane. The openings in the support plate enable gas or vacuum pressure to be applied to the flexible membrane where it covers each inlet channel opening, thereby deflecting the membrane and causing fluid inside the inlet channel and the flow channel to be moved by the deflecting membrane. In these embodiments, rhythmic up and down deflections of the flexible membrane cause fluid in the inlet channel to move up and down, and causes fluid in the connected flow channel to move back and forth.
32. The fluidic device of any one of embodiments 21-31, further comprising living cells adhered to a porous membrane that forms at least a portion of the bottom of a well of the multi-well cell culture plate.
33. A system for exposing living cells to fluid shear stress, which comprises a fluidic device according to any one of embodiments 1-20; a multi-well cell culture plate configured for use with the fluidic device; and means to cause fluid inside the horizontal section of the flow channel of the fluidic device to move. In some embodiments, the means to cause fluid inside the flow channel to move comprises a flexible membrane covering one or more of the outlet channel or inlet channel openings on the surface of the manifold body, and a device that causes pneumatic flexion, or hydraulic flexion, or mechanical flexion of the flexible membrane where it covers an outlet channel or inlet channel opening. In some embodiments, the device that causes pneumatic flexion is a membrane-flexion device that is configured to apply force to the flexible membrane directly below each of the inlet channel openings on the bottom surface of the manifold body.
34. The system of embodiment 33, wherein the multi-well cell culture plate comprises a porous membrane across the bottom of each well.
35. The system of embodiment 34, wherein living cells are adhered to the underside of the porous membrane of the wells of the multi-well cell culture plate.
36. The system of any one of embodiments 33-35, wherein the means to cause fluid inside the horizontal section of the flow channel of the fluidic device to move is selected from a pressure pump, a suction pump, flexible membrane disposed across an opening of the inlet channel or outlet channel of the fluidic device that can be deflected to move fluid, a bulb or balloon configured to apply suction or pressure to the fluid via the inlet channel or outlet channel of the fluidic device, and a mechanism to physically move the fluidic device in a reciprocating manner.
37. A method to apply shear stress to a living cell, which comprises adhering living cells to the bottom surface of a well of a multi-well cell culture plate, and fitting the multi-well cell culture plate to the top surface of the fluidic device of any one of embodiments 1-20. The multi-well cell culture plate is fitted to the top of the manifold body so that each well of the cell culture plate is inserted into one of the holes that extend downward into the manifold body from the top surface of the manifold body. The method can also comprise providing a fluid filling a flow channel inside the manifold body, so the fluid is in contact with the living cell, and applying a force to the fluid to cause the fluid to move, and thereby applying shear stress to the living cell. The fluid is typically a cell culture fluid, suitable to maintain the living cells.
38. The method of embodiment 37, wherein an effector substance is placed in at least one well of the multi-well cell culture plate.
39. The method of embodiment 37 or 38, wherein at least one flow channel of the fluidic device contains an aqueous cell culture medium.
40. The method of any one of embodiments 37-39, wherein a flexible membrane is applied to a surface of the manifold body to cover the openings where one or more outlet channel or inlet channel opens at the surface of the manifold body to form a fluid-tight seal with the manifold body, and a force is applied to the flexible membrane to induce motion of fluid in the flow channel and thereby produce shear stress on the living cells.
41. The method of any one of embodiments 37-40, wherein two wells on the multi-well cell culture plate have two different cell types adhered thereto.
42. The method of any one of embodiments 37-41, wherein two wells on the multi-well cell culture plate having two different cell types adhered to the undersides of the wells both intersect the same flow channel.
43. A device to expose living cells to fluid shear stress, wherein the device comprises: a plurality of wells having generally vertical walls and a generally horizontal floor, wherein at least a portion of the floor is a permeable membrane;
   at least one flow channel positioned below the wells so that the permeable membrane portion of the floor of each well separates the well from one of the at least one flow channels;
   an inlet that connects the flow channel to the exterior of the device, and an outlet that connects the flow channel to the exterior of the device, wherein a fluid path leading from the inlet, through the flow channel to the outlet passes beneath the permeable membrane portion of the floor of at least one well. Preferably, the plurality of wells are connected together by a generally horizontal plate in the form of a multi-well cell culture plate, and the multi-well cell culture plate can be separated from the remainder of the device.
44. The device of embodiment 43, wherein the permeable membrane is adapted for living cells to adhere to the underside of the permeable membrane. In many embodiments, the permeable membrane is part of a multi-well cell culture plate that can be separated from the remainder of the device.
45. The device of embodiment 43 or 44, wherein each well has a volume between about 0.001 mL and 0.5 mL. Preferably, the wells are arrayed in a configuration that matches conventional high-throughput screening devices, such as the 96-well Transwell® plates described herein.
46. The device of any one of embodiments 43-45, wherein living cells are adhered to the underside of the permeable membrane forming the floor of at least one of the wells.
47. The device of any one of embodiments 43-46, which comprises at least two separate flow channels, wherein each of said flow channels is separated by the permeable membrane portion of the floor of a well from one well, or two wells, or three wells.
48. The device of any one of embodiments 43-47, which comprises two modular components, where the first modular component comprises the generally vertical walls and the generally horizontal floor of the wells of said device. The first modular component can comprise a multi-well cell culture plate, e.g., a Transwell® plate as described herein. Optionally, the first modular component further comprises a manifold structure having holes that extend through a rectangular cuboid solid, wherein the holes are sized and configured to accept the wells of the multi-well cell culture plate.
49. The device of embodiment 48, wherein the second component comprises one or more recesses in one of its outer surfaces that form said at least one flow channel when the first component and second component are assembled together.
50. The device of any one of embodiments 43-49, which comprises at least 32 wells, or at least 48 wells, or at least 120 wells, or at least 500 wells.
51. The device of any one of embodiments 43-50, which comprises at least four separate flow channels, or at least 32 separate flow channels, or at least 120 separate flow channels, or at least 500 separate flow channels.
52. The device of any one of embodiments 43-50, which comprises at least four separate flow channels, or at least 32 separate flow channels, or at least 120 separate flow channels, or at least 500 separate flow channels.
53. A method to use the device of any one of embodiments 43-51, which comprises placing living cells on a permeable membrane of the device, and contacting the living cells with fluid moving through a flow channel within the device. In this embodiment,
54. A system comprising the device of any one of embodiments 43-52, and means to cause fluid inside the horizontal section of the flow channel of the fluidic device to move.

### Manifold Body

The manifold body of the fluidic devices disclosed herein is typically a block of solid, water-impermeable material with flow channels formed therein as described in the embodiments above, and having at least two and preferably at least four holes extending downward from its top surface. Generally, the manifold body is block-like (cuboid) in shape, having a substantially horizontal top surface and a substantially horizontal bottom surface and, often though not necessarily, generally square or rectangular horizontal cross-sections. Typically, the manifold body is a rectangular cuboid, wherein each face is generally rectangular in shape, with optional extensions or protrusions as described herein.

The size of the manifold body can be selected to suit a particular application or purpose. In some embodiments, the horizontal dimensions of the manifold body are sized to accommodate a multi-well cell culture plate of a desired size, such as a commercially available cell culture plate having 4, 8, 16, 32, 96, 384, or 1536 wells. In some embodiments, the manifold body is sized to accommodate a 96-well plate having the configuration of a 96-well Transwell® plate as described herein.

The holes that extend downward from the top surface of the manifold body are configured to receive wells of a multi-well cell culture plate. The size and placement of the holes can be adapted to match and mate with a particular multi-well cell culture plate, or vice versa. The holes can be of any suitable shape, but are often round, oval, square or rectangular in horizontal cross-section; corners of the square or rectangular holes are optionally rounded, which can simplify manufacture or improve performance. In certain embodiments, the holes are round in horizontal cross-section. The hole shape and size may be selected to accommodate a desirably configured multi-well cell culture plate, such as ones described below.

The manifold body also comprises two or more separate flow channels. The flow channels are positioned and configured to have a horizontal section that intersects with the holes described above, extending downward from the top surface of the manifold body. Each flow channel is thus shaped and positioned within the manifold body so its horizontal section that intersects one of the holes is at a depth, i.e., at a vertical distance below the horizontal top surface of the manifold body, so that a well of a multi-well cell culture plate, when inserted into the hole, reaches the flow channel so the bottom of the well, when the cell culture plate is fitted to the manifold body, is in contact with fluid in the horizontal section of the flow channel when that section of the flow channel is filled with fluid. The depth of the flow channel below the top surface of the manifold body thus corresponds to the length of a well (the length by which the well extends downward from the horizontal plate) of the multi-well cell culture plate that the manifold body is designed to be used with. The flow channels do not necessarily need to be all at one depth, e.g. higher density of testing units (discussed below) may be achieved with designs that have flow channels at different depths. In a preferred embodiment, however, the wells of the multi-well cell culture plate are of uniform length, and the flow channels are thus at a uniform depth below the top surface of the manifold body.

The device comprises at least two separated flow channels, and optionally comprises a number of flow channels that is equal to the number of holes extending downward from the top surface of the manifold body, which in turn is typically equal to the number of wells of the multi-well cell culture plate that the manifold body is designed to accommodate. Thus in one embodiment, the fluidic device has one flow channel for each hole in the manifold body. For some purposes, however, it is advantageous to have two or more wells in contact with the fluid of a single flow channel. Accordingly, in some embodiments, one or more of the flow channels in the fluidic device of the invention intersects at least two and optionally three or four or more than four of the holes that extend downward into the manifold body, and thus a single flow channel can be in contact with two, three, four or more than four wells of a multi-well cell culture plate when said cell culture plate is fitted onto the manifold body.

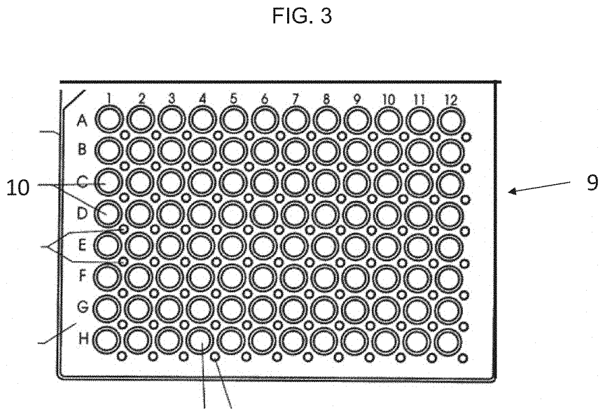

While the horizontal section of the flow channels can be oriented in any suitable direction, in some embodiments they are aligned at a 45-degree angle relative to the rows of holes extending downward from the top surface into the manifold body. In other embodiments, the horizontal sections of the flow channels are aligned parallel to the rows of holes. FIG. 3 depicts the top of a 96-well multi-well cell culture plate and illustrates why this arrangement can be advantageous. Some commercially available multi-well cell culture plates such as the Corning Transwell® plate shown in FIG. 3, have an opening for each well that is positioned on a 45-degree angle relative to the right-angle ‘row and rank’ alignment of the holes in the manifold body top surface. The hole provides access through the top plate of the multi-well cell culture plate, permitting fluid, for example, to be added to an opening of an outlet for the flow channel from above the cell culture plate when it is mounted on top of the manifold block. As a result, the opening in the cell culture plate can be used to insert cell culture fluid in to the flow channel via the opening of the outlet channel through this opening in the cell culture plate, in configurations where the opening of the outlet channel is also positioned at a 45-degree angle relative to the rows and ranks of holes in the manifold body. When this opening in the cell culture plate is utilized, it logically leads to a configuration in which the flow channel going under a well is also aligned at the same 45 degree angle so that the horizontal section of the flow channel running under the adjacent hole runs straight across the bottom of the hole (and the bottom of the well of an inserted cell culture plate) when aligned at a 45-degree angle. See FIG. 3, showing a small hole for the outlet channel on the diagonal between the larger holes (6) configured to receive the wells of a multi-well cell culture plate. This alignment is thus a preferred way to arrange the flow channels and outlet openings for a 96-well embodiment of the manifold body that is adapted to be used with the Corning Transwell® plates.

Each flow channel intersects at least one hole and is thus in fluid contact with at least one well of a multi-well cell culture plate when the cell culture plate is fitted onto the manifold body. The well intersects with a horizontal section of the flow channel, and the width of the flow channel is generally similar to the width of the well it contacts in the zone where the two features intersect. In some embodiments, the well is slightly wider than the flow channel in this section: this permits the bottom of the well to be consistently positioned right at the depth of the ceiling of the flow channel, even if there is slight variation in the length of the wells of the multi-well cell culture plate. This can reduce turbulence in the fluid flowing inside the flow channels that otherwise could arise right in the area where fluid shear stress will be experienced by cells on the bottom of the well of the multi-well cell culture plate, and thus this design can contribute to consistent shear stress, better performance and more reliable results when using the fluidic device to expose such living cells to shear stress from fluid flow. The horizontal section of the flow channel is generally rectangular in its vertical cross-section, though its shape is not necessarily critical. The vertical cross-section of the flow channel generally has a height of 0.1 to 10 mm, depending on the size of the manifold body and the dimensions of the wells of the cell culture plate with which the manifold body is designed to be used. For example, a 96-well cell culture plate sized like the Corning Transwell® plates would typically have a flow channel with an interior height of 1-5 mm, and a width of 5 to 15 mm. The dimensions and shape of the flow channels are further discussed below.

The vertical height of the manifold body can be selected to suit a particular purpose or application, and typically is greater than the sum of the depth of the wells of a multi-well cell culture plate chosen for use with the manifold body plus the vertical depth of the horizontal section of a flow channel inside the manifold body: the vertical height of the manifold body is greater than this sum because the manifold body comprises solid substrate below the flow channel sufficient to contain fluid flow within the flow channel and maintain structural integrity of the fluidic device during manufacture and operation.

A testing unit as used herein comprises a well provided by a multi-well cell culture plate fitted onto the manifold body, so that the well is inserted into one of the holes that extend downward from the top surface of the manifold body, plus the horizontal section of the flow channel that particular hole intersects; thus it includes the interface region where fluid flowing through the flow channel contacts cells adhered to the bottom of the well. Optionally, the testing unit comprises living cells either inside the well or on the outside surface of the permeable membrane of the bottom of the well; optionally living cells can be both inside the well and on the bottom of the outside of the well, in which case the two separate groups of living cells within a single testing unit can be different cell types, and the testing unit can address interactions between the two different cell types. FIG. 2 is a schematic side view of one testing unit of an embodiment of the fluidic device of the invention.

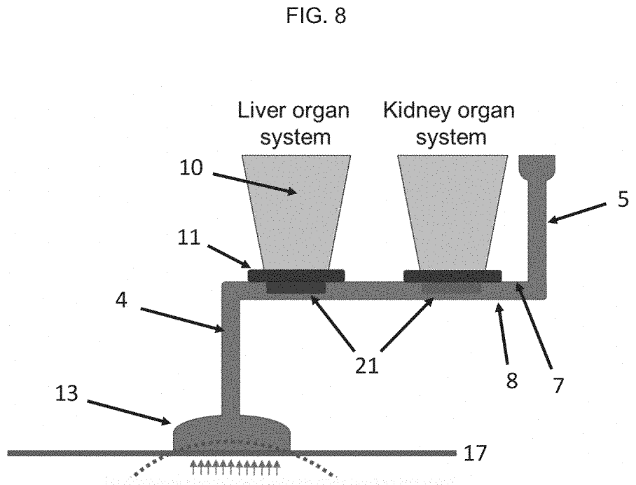

As described below, a flow channel may intersect with two or more of the holes and thus may be in fluid contact with two or more wells when the cell culture plate is fitted to the manifold body. However, since each of the two wells can have a different fluid inside and/or a different population or type of cells inside the well or on the bottom of the outside of the well, each well exposed to a flow channel is viewed as a distinct testing unit. Thus the fluidic device of the invention comprises two or more, often four or more, and optionally 96, 384 or 1536 (or more) separate testing units within a single fluidic device. Note a single flow channel can serve two or more wells as further described herein, so there need not be one flow channel per well. Also, the inlet channel and/or outlet channel for a flow channel may functionally serve more than one testing unit, so there need not be an inlet channel and an outlet channel for each testing unit, even though each testing unit is connected to and served by an inlet channel and an outlet channel. In these embodiments, the flow channel will typically intersect two adjacent holes (and wells when a multi-well cell culture plate is fitted to the manifold body), and will have an inlet channel on one side of the two adjacent holes and an outlet channel on the other side of the adjacent holes. See e.g. FIG. 8. Note that adjacent holes (or wells) can be holes in a single rank or file of an array, or they can be diagonally adjacent holes or wells.

The fluidic device of the invention, or individual components thereof, can be made from any suitable materials, such as PDMS (Polydimethylsiloxane), glass, PMMA (polymethylmethacrylate), PET (polyethylene terephthalate), Polyetherimide (ULTEM), SILTEM, poly ether ether ketone (PEEK), PC (Polycarbonate), thermoplastic polyurethane (TPU), silicon, silicone, PE (polyethylene), PS (polystyrene), ABS (acrylonitrile-butadiene-styrene), etc., stainless steel, titanium, aluminum, ceramic, or a combination thereof. Ideally, the manifold body of the device of the invention is composed of materials that can be autoclaved for sterilization so it can easily be adequately cleaned for re-use. A preferred material for the manifold body for this device, when autoclave sterilization is contemplated, is thermoplastic polyurethane (TPU).

### Multi-Well Cell Culture Plate

The multi-well cell culture plate referred to herein and used with the fluidic device of the invention is a rigid or semi-rigid horizontal plate having a plurality of bucket-like wells extending downward from the horizontal plate. The horizontal plate is often transparent or partially so to promote observation of the operation of the device, and is frequently made of a plastic material such as PET, PVC, nylon, polycarbonate, polyethylene, polypropylene, polyester, polystyrene, or a polymethacrylate, or a copolymer or blend such as ABS (acrylonitrile butadiene styrene), PC/ABS, or PE/ABS, or combinations of these. The wells have walls that extend downward from the horizontal plate resembling the sides of a bucket, and a bottom extending across the lower end of the sides to form a bottom of the well or ‘bucket,’ so that the well encloses a volume suitable for the purpose of the cell culture plate, and each well of the cell culture plate is fluidically independent of or separate from every other well. This ensures that each of the testing units can be used independently.

The number and size of the wells can be chosen to suit a particular purpose or application. Preferably, the bottom of the well comprises or consists of a permeable or porous membrane suitable as a support substrate for growing cells, which may grow on the inside of the well or on the outside of the bottom of the well. The permeable membrane is particularly useful when cells are to be grown on the bottom of the well, i.e., outside the ‘bucket’ of the well, and its permeability should be such that water or cell culture medium placed inside the well can reach and hydrate and/or nourish the living cells adhered to the bottom of the outside surface of the well.

While the dimensions and spacing of wells in these cell culture plates can be adapted to a wide variety of purposes, typically the cell culture plate will have from 4 to 4000 wells, each having a volume between 0.01 mL and 5 mL, and having a permeable membrane on the bottom of each well with an area of at least 0.1 mm² and commonly an area of at least 1 mm². Frequently, in order to enhance compatibility with conventional apparatus and/or processing systems and protocols for sample or data handling, the fluidic device is designed and configured for compatibility with a 4-well, 8-well, 16-well, 32-well, 96-well, 384-well or 1536-well cell culture plate.

The design and construction and materials for such multi-well cell culture plates are well known in the art; indeed, certain such cell culture plates are commercially available, e.g., the Transwell® plates from Corning, made of treated polycarbonate or treated PET. Suitable materials for the multi-well cell culture plates for use with the fluidic device of the invention include PE, polypropylene, polycarbonate, PET, and the like, including treated versions of these materials, i.e., TC-treated or tissue culture treated materials. In certain embodiments, to take advantage of such commercially-available cell culture plates, the manifold body may be sized and configured to receive the wells of a particular type of cell culture plate like a 96-well Transwell® plate, e.g., Corning parts number C3391, C3381 C3380, C3386, C3385, C3387 or C3388. These cell culture plates have a 96-well format with an 8×12 array of identical wells that are circular in horizontal cross section, cylindrical or approximately cylindrical in shape, and about 1.0 cm in depth (the vertical dimension of the well that extends downward from the horizontal top plate), having a well volume of 0.23 mL per well, and a permeable membrane of 4.26 mm diameter spans the bottom of each well and is suitable as a substrate for growing cells inside the wells or on the bottom of the outside of the wells.

The wells in such plates are typically evenly spaced within the array: in the Transwell® plates described above, for example, the ranks and rows of wells are spaced apart by about 0.9 cm (0.8 to 1.0 cm) from center to center, and the wells have an outer diameter of about 0.5 cm (0.4 to 0.6 cm). This arrangement of wells provides a suitably high density of testing units for high-throughput testing. FIG. 3 depicts a multi-well cell culture plate of this configuration.

### Sealing Surfaces

The device of the invention includes a sealing surface to provide a liquid-tight seal between a surface of the manifold body and a surface of a multi-well cell culture plate. The sealing surface can be made of a different material from the manifold body and/or culture plate, and can be any material and any form that provides a fluid-tight seal to maintain the internal fluid volumes of the manifold body separated within the manifold body, to avoid fluid loss, and to prevent fluid inside the flow channels of the manifold body from escaping the device or leaking into the interior of the wells of the multi-well cell culture plate when one is present. In particular, a sealing surface is selected and positioned to provide a fluid-tight seal between the manifold body and each well of the multi-well cell culture plate, so that fluid contained in the flow channel inside the manifold body does not escape around or into the well of the multi-well cell culture plate, even when the fluid in the flow channel is moving to provide shear stress across the bottom of the well(s) of the multi-well cell culture plate. In certain embodiments, the sealing surface ensures that a fluid in the flow channel contacting living cells on the bottom or the exterior of a well of the cell culture plate flows within the flow channel(s) in the manifold body from inlet to outlet (or vice versa), and does not escape via the hole around the well. Preferably, the sealing surface is configured so that the seal between the manifold body surface and a well of the cell culture plate does not create turbulence around the well, or interfere with the flow of fluid in the channels in the manifold body as it flows past living cells on the bottom surface of the wells, so that the fluidic device can provide a consistent fluid shear on the living cells.

In some embodiments, the sealing surface is a gasket or similar structure that can be interposed between surfaces of the manifold body and surfaces of a multi-well cell culture plate when one is fitted to the top surface of the manifold body. It can thus be a flat gasket that sits atop the manifold body with holes through the gasket, so the multi-well cell culture plate can be fitted with its wells inserted into the holes in the manifold body. In some embodiments, the sealing surface is an O-ring or a sleeve that fits into each of the holes of the manifold body, and typically would be sized to fit snugly against the inner surface inside each of the holes extending downward from the top surface of the manifold body, or to snugly fit onto the outer surfaces of each of the wells of the multi-well cell culture plate, so the sealing surface provides a fluid-tight seal when the multi-well cell culture plate is fitted to the manifold body so that the wells of the cell culture plate extend downward inside the holes when the two parts are assembled. The sleeve or O-ring, which can be integrally formed inside the holes or on the outside of the wells, or can be an independent component made of the same or different material from the wells and the manifold body, forms a seal between a surface of the manifold body, particularly a surface on the inside of the holes extending downward from the top surface of the manifold body, and an outer surface of the multi-well cell culture plate, usually the outer surface of each well. These sealing surfaces can be separate components that are removable, and can be made of any suitable material compatible with an aqueous environment such as a culture fluid used for maintaining live cells.

In one embodiment, the manifold body provides a sealing surface lining a portion of each of the holes that extend downward from the top surface into the manifold body. Suitably, this sealing surface can be an O-ring positioned inside each of these holes, preferably at or near the lowest portion of the hole, i.e. at or just above the level where the hole intersects with a flow channel. The O-ring can be sized to snugly fit inside these holes; in some embodiments, there is a horizontal groove inside each of the holes near the bottom of the hole, positioned typically just above the level where the hole intersects a flow channel inside the manifold body. This placement minimizes ‘dead space’ in the fluid-holding volume inside the manifold body of the device: the fluid-holding volume comprises the horizontal section of the flow channel and optionally at least part of the inlet channel and/or outlet channel connected to each flow channel.

### Multi-Piece Manifold Bodies

The manifold body can be made as a single block, such as by boring openings into the block to form the holes that extend downward from the top surface and to form the flow channels and their connected inlet channels and outlet channels, or 3-D printing the manifold with the required flow channels, inlet channels, and outlet channels. However, for convenience of manufacture and/or operation, in a preferred embodiment, the manifold body can comprise two or more than two portions that are held or connected together.

In some embodiments, the manifold body can be made in two or more parts to provide ease of manufacture, operation, and cleaning for re-use. In some embodiments, the manifold body of the device is sliced horizontally into layers that be manufactured layer by layer and stacked one on top of another for assembly into a manifold body. These embodiments are referred to as horizontally bisected manifold bodies, or as a two-piece, three-piece, or four-piece manifold body, for example, to indicate the number of layers to be separately made and stacked together to form a manifold body. Each layer of these embodiments is referred to in the following description as a ‘section’ of the manifold body.

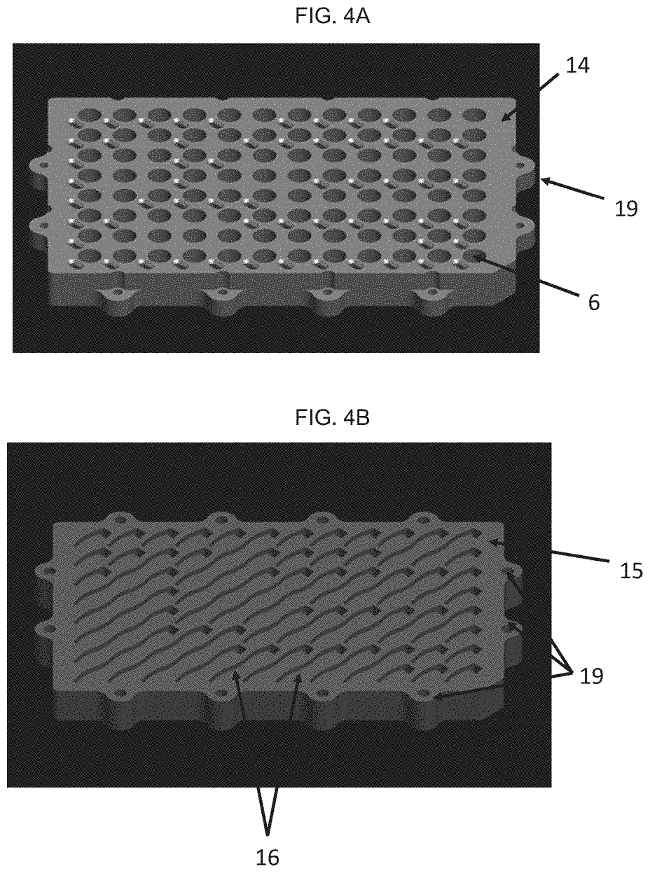

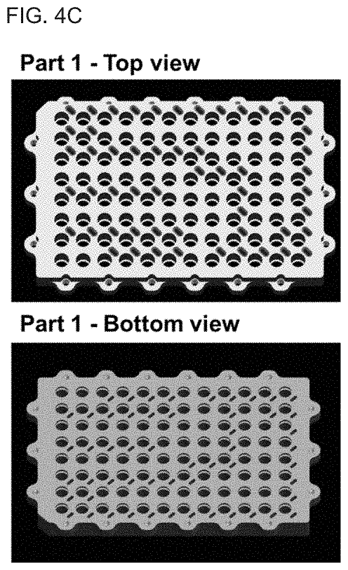

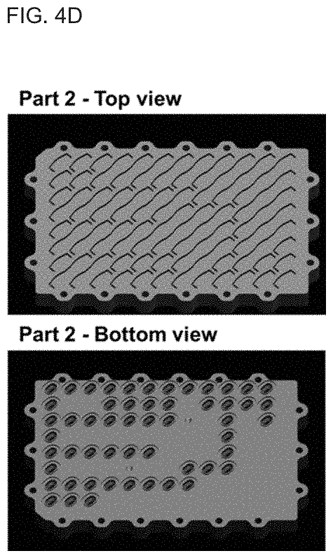

The manifold body can be sliced horizontally in several different ways to simplify manufacture or assembly or operation. In some embodiments, the manifold body comprises two layers (two portions), and is referred to as a two-piece manifold body. In such embodiments, the manifold body comprises an upper portion and a lower portion, and is typically bisected horizontally at either the top level (ceiling level) of the horizontal section of the flow channels inside the manifold body, or at the bottom level (floor level) of the horizontal section of the flow channels. FIGS. 4A and 4B depict upper and lower portions of a two-piece manifold body bisected at the level of the ceiling of the flow channels.

In the two-piece embodiments, either the lower surface of the upper portion (layer) of the horizontally bisected manifold body (FIG. 4A), or the upper surface of the lower portion (FIG. 4B) of the horizontally bisected manifold body can be generally flat, which minimizes manufacturing complexity. For example, in this embodiment, the holes extending downward from the top surface of the manifold body extend entirely through the upper portion, and do not enter the lower portion at all. This avoids the need to precisely bore a hole to the depth of the flow channel in one portion of the manifold body, or to exactly align these holes in the upper portion with counterpart or continuation of the holes in the lower portion. The other of those two surfaces (either the lower surface of the upper portion of the horizontally bisected manifold body, or the upper surface of the lower portion of the horizontally bisected manifold body-see FIG. 4B) can be machined, cast, or otherwise embossed with canal-like recesses (16) formed in the generally flat surface: these recesses then form the horizontal sections of the flow channels when the two portions or layers of the horizontally bisected manifold body are assembled together, and optionally, when suitable placed, such recesses can also be used to form inlet channels and/or outlet channels for the flow channels.

In either one piece or two-piece manifold body designs, the manifold body optionally comprises a groove around each of the inlet channel openings in the bottom surface of the manifold body, wherein each said groove is configured to hold an O-ring that is positioned to form a seal between the manifold body and the surface on which it sits when the fluidic device is assembled, e.g., the O-ring can be positioned to form a seal between the manifold body and a flexible membrane when the manifold body is placed on a flexible membrane, thus providing a seal around each of the inlet openings. This can improve the seal around the inlet opening when the device is operated by applying pressure and/or suction to the underside of the flexible membrane, thereby driving fluid motion in the fluid channel as illustrated in FIG. 2. This ensures that each flow channel is effectively separated from the others.

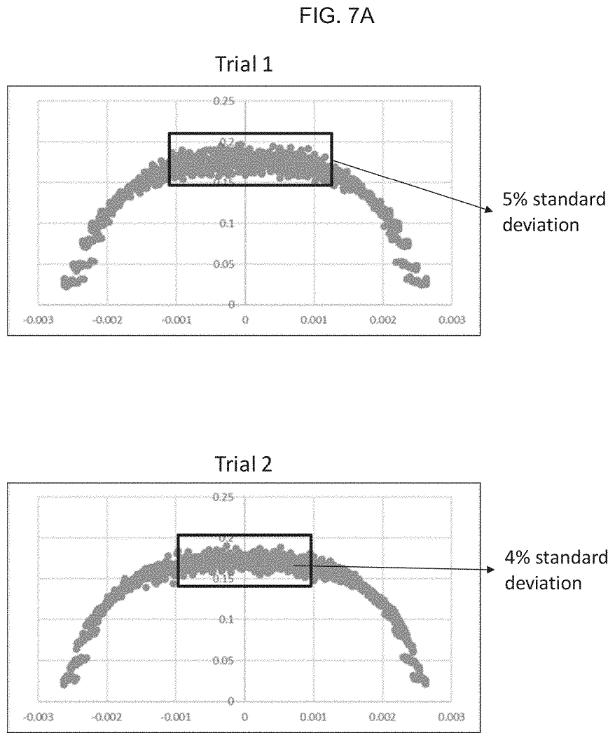

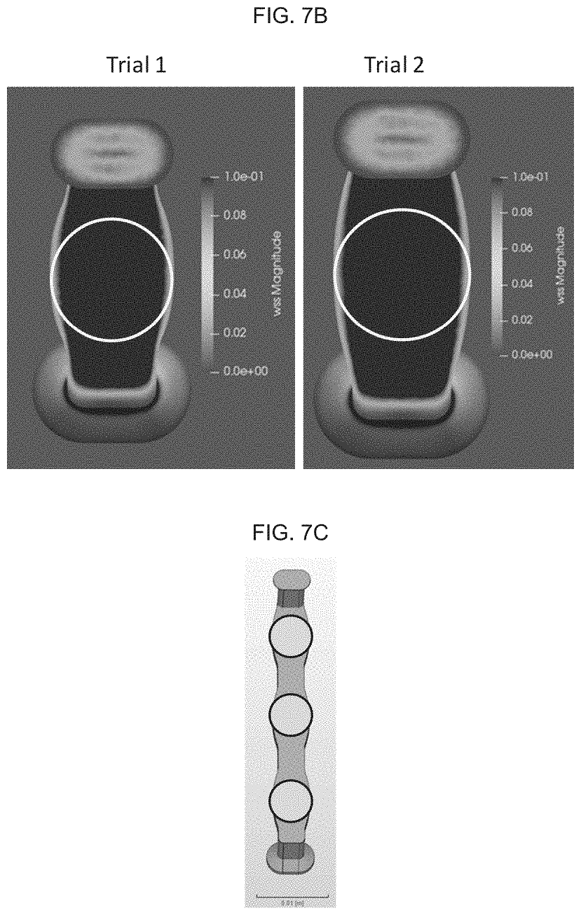

In FIG. 4A, the holes (6) configured to accept wells of a multi-well cell culture plate are arrayed in an 8×12 pattern, and small extensions or ears (19) are provided to allow the portions or layers of the device to be connected securely together, as shown in the exploded view (FIG. 5A) of an embodiment of the fluidic device. FIG. 5B is an exploded view of an embodiment having additional set of O-rings (11b) to form a seal between the underside of the manifold body (15) and the flexible membrane (17) when the device is assembled. The canal-like recesses (16) in the upper surface of the lower portion of the manifold body (FIGS. 4B and 4D) are aligned on a diagonal arrangement so that each hole through upper portion (14) intersects one of the canals (16, FIGS. 4B and 4D) that form flow channels when the device is assembled. The canals extend across one, two or three of the holes (6) through the upper portion. Optionally, the canals that form the flow channels are shaped to provide uniform shear stress across the bottom of the hole (6) and thus across the bottom of a well inserted into the hole, according to fluid dynamic simulations discussed below. See, e.g. FIGS. 7A, 7B, and 7C.

In addition, the horizontally bisected manifold body design can greatly simplify inserting components or materials of interest inside the manifold body. For example, where the sealing surface described herein is provided by O-rings set inside the holes that extend downward from the top surface of the manifold body, depending on the size and depth of the holes, inserting such O-rings from the top surface of the manifold body could be challenging, especially if the O-rings are placed at or near the level where the hole intersects the horizontal section of a flow channel, which may be well below the top surface of the manifold body. A horizontally bisected manifold body design can simplify inserting the O-rings at or near the depth at which the holes intersect with the flow channels, for example, as the O-rings (11a) can be inserted from the lower surface of the upper portion of a two-part horizontally bisected embodiment of the manifold body. See FIGS. 5A and 5B.

The upper and lower portions of the two-part manifold body can be held together mechanically, e.g. by screws or similar fasteners, or by a clamping device that presses them together, or by an adhesive. In some embodiments, the two portions are held together by screws or nuts and bolts. For simplicity, the description herein will refer at times to using screws to hold components or layers of the manifold body together, while encompassing the use of nuts and bolts as well. The skilled practitioner will understand that selection of the preferred faster for such assemblies depends on the specific materials used and the size of components, etc., and use of the term ‘screws’ simplifies discussion without limiting the explanation or disclosure specifically to use of screws. Optionally in these embodiments a gasket can be provided between the upper portion and lower portion to provide a fluid-tight seal. Screws can extend through either of the two portions into the other portion, or small ears (19) can be provided around the perimeter of both the upper and lower portions, where the ears are aligned vertically so that a screw or bolt (20) can extend through an ear on one portion into or through a corresponding ear on the other portion to fasten the two portions together. See FIGS. 5A and 5B. Alternatively, the upper and lower portions of the two-piece manifold body can be held together with an adhesive. Any suitable adhesive can be used for this purpose. In some embodiments, the two portions are glued together using volatile solvents such as acetone, hexane or E6000 glue (styrene-1,3-butadiene polymer in tetrachloroethylene) or B482 Reltek® adhesive (a semi-flexible two-part epoxy resin).

Typically, the upper portion of this embodiment is flat with a uniform thickness, where the upper surface of the upper portion is the top surface of this embodiment of the manifold body, while the lower surface of the upper portion is generally flat and parallel to the top surface of the upper portion. Similarly, the lower portion of this embodiment is generally flat with a uniform thickness, where the upper surface of the lower portion is the top surface of this embodiment of the manifold body, while the lower surface of the upper portion is generally flat and parallel to the top surface of the upper portion. Optionally, the holes in the top surface of the manifold body extend entirely through the top portion of the manifold body and do not extend into the lower portion of the manifold body. In these embodiments, the lower portion of the manifold body has open recesses (canals) formed in its upper surface, so that when upper and lower portions of the two-piece manifold body are fitted together, the bottom surface of the upper portion of the manifold body forms a ceiling over the open recesses, thereby forming the flow channels in the manifold body. Preferably, the holes in the upper portion are sized and positioned so that when the upper portion is fitted to a multi-well cell culture plate, the bottoms of the wells lie flush with the bottom surface of the upper portion of the two-piece manifold body; and the open recesses in the upper surface of the lower portion of the two-piece manifold body are positioned and sized to provide a flow channel that traverses across all or nearly all of the bottom of each well of the multi-well cell culture plate.

In these two-piece manifold body embodiments, the sealing surface can be a sleeve or an O-ring lining each of the holes that receive wells of the multi-well cell culture plate. Preferably, these holes are round in cross-section. Sleeves can be inserted into the holes from the top or bottom of the upper portion. O-rings can readily be fitted inside the holes through the upper portion of the horizontally bisected (two-piece) manifold body, optionally in a groove formed around the perimeter at or near the lower end of each hole (12, in FIG. 1) formed through the upper portion of the two-piece manifold body and just above where the hole intersects its flow channel.

FIG. 5A shows a two-piece manifold body with a layer of O-rings (11a) to provide the sealing surfaces of the device to seal against outer surfaces of wells of an inserted Transwell® plate (not shown), and underneath the lower portion (15) of the two-piece manifold body is a flexible membrane (17) that can be flexed to drive fluid motion within the flow channels. Below the flexible membrane is a support plate (18) to hold the membrane firmly against the manifold body. This support plate is optional, and may not be needed when the flexible membrane (17) is adhered to the lower portion (15) of the manifold body. FIG. 5B shows another embodiment, which is similar to the one in FIG. 5A but includes an additional set of O-rings (11b) that can be used to form a better seal between the lower portion of the two-piece manifold body (15) and the flexible membrane (18) if needed.

Another aspect of the invention provides a method to remove a multi-well cell culture plate from the manifold body of the device of the invention by utilizing bolts or screws. In embodiments where the sealing surface is an O-ring or sleeve inserted into each hole, once a multi-well cell culture plate is fitted to the top surface of the manifold body and pressed firmly into place to make secure, fluid-tight seals between each of the wells and the hole it fits into, the cell culture plate can be so firmly seated that separation of the cell culture plate from the manifold body can be difficult.

Thus, in some embodiments of the manifold bodies of the invention, two or more screws are positioned in the upper portion of the manifold body so they can be used to apply pressure to the underside of the cell culture plate, so they can be used to push the cell culture plate off the manifold body when it is necessary to remove the cell culture plate. In some embodiments, 4, or 6, or 8, or 10, or 12, or more than 12 screws are positioned for this purpose. Small extensions (‘ears’) around the perimeter of the manifold body, often around the perimeter of the upper portion of a two-piece manifold body, can be threaded to receive screws that are positioned and sized so that, when the screws are advanced, they apply pressure to the underside of the cell culture plate, or to extensions on the cell culture plate, and thereby push the cell culture plate off the manifold body. Alternatively, threaded holes can be provided through the manifold body, or through the upper portion of a two-piece manifold body, so that screws can be inserted and advanced through the threaded holes to apply pressure to the underside of the cell culture plate and push the cell culture plate off the manifold body.

### System for High-Throughput Testing on Living Cells Exposed to Fluid Shear

In one aspect, the present disclosure provides a fluidic device as described above as part of a system for exposing living cells to shear stress. The system comprises, in addition to a device of the invention, a multi-well cell culture plate that can be fitted into or onto the manifold body described above. Optionally, the system further comprises a means for moving fluid within the flow channels of the manifold body. This means for moving fluid within the flow channels can be any suitable means, such as a pump or a mechanism for applying suction or force to a fluid in the flow channels. The fluid movement can be flow through the manifold body, or it can be a reciprocating motion where the fluid remains largely or entirely within the flow channel but is moved back and forth within the horizontal section(s) of the flow channel. Embodiments providing for reciprocating motion of the fluid are especially useful as they permit the use of a minimal volume of fluid that remains in contact with the living cells for a potentially prolonged period of time. This permits the cells to act on or interact with materials in the fluid over time frames similar to those that occur in vivo for many of the types of cells of interest for use with the devices and systems described herein. For example, metabolites from the cells can accumulate in the fluid, or material in the fluid can be slowly metabolized or utilized by the cells, simulating the interaction such cells would have with fluids in their natural environment.

The fluidic device is designed to expose living cells to two different fluid sources: while the cells grow in contact with a moving fluid medium that provides shear stress on the cells to simulate the in vivo conditions that some cell types normally face (the moving fluid in the flow channels in the manifold body), they can also be nourished by or stimulated by fluid inside the wells of the multi-well cell culture plate. While it does not form an impenetrable barrier, the porous membrane of the bottom of each of these wells, combined with the matrix of cells growing on the surface of this porous membrane, substantially separates the fluid inside the well from the fluid in the flow channels inside the manifold body; yet both fluids can supply nutrients and/or effectors to the living cells, and both fluids can be affected by the surfaces of these living cells or by materials passing into and out of the living cells.

For example, cells in the kidney tubule are subjected to continuous fluid motion at their surface, and it has been shown that this fluid shear has important effects on their surface characteristics and internal biochemistry, as discussed above. The devices of the invention are of special value and interest for studying cells that are typically exposed to shear forces in vivo, and particularly for testing

The cells to be exposed to shear stresses are typically grown on the underside of the porous or permeable material forming the bottom of a well in a multi-well cell culture plate, rather than on the upper surface of that porous material; i.e., they are actually growing on the outside of the wells rather than inside the wells where they would normally be for applications of such multi-well cell culture plates. However, it is also possible to grow cells inside the wells instead of, or in addition to having living cells on the bottom surface of the well, thus the device enables a user to concurrently test cells exposed to shear adjacent to cells that do not experience shear stress, further enhancing the flexibility and utility of the device.

In use, typically an aqueous solution is put into a well of the cell culture plate: an effector can be added to this aqueous solution, which may or may not also contain nutrients. Living cells are adhered to the underside of the porous membrane forming the bottom of the well. Optionally, the well can contain cells inside the well that are the same type as the cells on the underside of the porous membrane, or cells of a different type from those on the underside of the porous membrane that forms the bottom of the well. Fluid in the well can diffuse into the cells on the underside of the porous membrane to deliver nutrients, effectors, or both.

The fluid in the flow channel is directly in contact with living cells growing on the underside of the porous membrane, and typically this fluid contains suitable nutrients to support those cells; optionally, an effector can be added to this fluid in addition to or instead of putting an effector inside the well(s). The layer of cells on the underside of the porous barrier provides a substantial barrier that separates the fluid inside the well from the solution contained in the flow channel below the wells of the cell culture plate, though the barrier is not impenetrable, as materials can enter the cells from one space and leave the cells in the other space.

The solution in the flow channel can be used to simulate an in vivo flowing liquid: motion can be imparted to the fluid in the flow channel to apply shear stress on the surface of the cells growing on the porous surface. This creates an environment that better simulates certain tissues or organs than a static environment and thus better represents the cells in vivo than a static environment would.

The desired motion of the fluid in the flow channel, and the associated shear stress produced by this fluid, can be produced in a variety of ways. For example, fluid can be pumped into the flow channel via its inlet, and can flow through the horizontal section of the flow channel and out of the flow channel via the outlet. The fluid in such embodiment can be recirculated, so the same fluid flows through the flow channel repeatedly. Alternatively, fluid pumped through the flow channel may be collected for analysis, or discarded, without being recirculated.

In some embodiments, the flow channel is filled or partially filled with fluid, so that at least the horizontal section of the flow channel that intersects the hole(s) in the manifold body is filled with fluid. This places the fluid in contact with living cells on the underside of the well(s) of a multi-well cell culture plate when such plate is fitted to the upper surface of the manifold body with the wells of the plate inserted into the holes in the manifold body. In these embodiments, a reciprocating motion can be used to move the fluid back and forth across the living cells to provide shear, without constantly changing or re-supplying the fluid or recirculating it through a pump. This arrangement can provide consistent levels of shear to the living cells, even though the direction of motion of the fluid flow reverses periodically due to the reciprocating motion of the fluid in the flow channel.

Reciprocating motion of the fluid can be induced in any suitable manner. It can be produced by moving the manifold back and forth in a direction parallel to the horizontal section of the flow channel. For this method, both inlet and outlet channels can extend upward from the horizontal section of the flow channel, so the fluid inside the flow channel sloshes back and forth in the channel, and by introducing a suitable amount of fluid into the flow channel so that at least some fluid remains in both inlet and outlet channels during motion, and selecting an appropriate rate of lateral motion, the cells are exposed to reciprocating fluid motion with minimal exposure to air or turbulence. Similarly, the manifold body can be tilted back and forth (“rocked)” along the axis parallel to the horizontal section of the flow channel to produce similar reciprocating motion.

The reciprocating motion can also be imparted by applying increased pressure or reduced pressure to the fluid in the flow channel via the inlet channel opening or the outlet channel opening, or both. In one such embodiment, the inlet is a vertical tube that opens at the bottom surface of the manifold body, and reciprocating motion is imparted by applying increased pressure or decreased pressure to the fluid at the opening of the inlet channel or the outlet channel. This may be used to simulate a system where the cells are affecting the fluid or its contents, e.g., it would permit the cells to metabolize a component in the flow channel fluid, or allow an effector compound or its metabolite from the well to gradually build up in the flow channel fluid. While the cell layer provides a reasonably effective barrier between the fluid in the well and the fluid in the flow channel, at least some substances can slowly diffuse between the fluids by passing through the living cells.

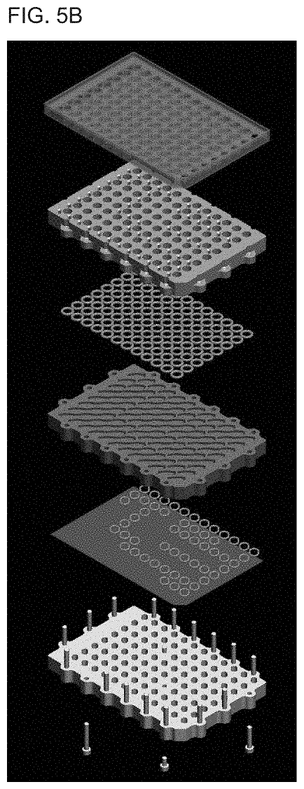

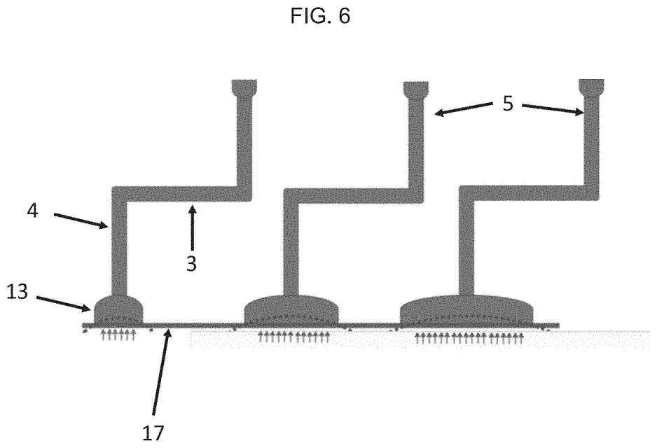

In one embodiment, a flexible membrane is applied to the bottom surface of the manifold body to form a liquid-tight seal with the manifold body, so the flexible membrane extends across each inlet channel opening or each outlet channel opening that opens to the bottom surface of the manifold body. Because the membrane is flexible, pressure applied to the membrane pushes the fluid in the inlet upward, resulting in movement of the fluid in the horizontal section of the flow channel. See FIG. 6, illustrating how enlarging the opening (13) where the inlet channel opens to the exterior of the manifold body at its lower surface can increase the amount of fluid motion produced by flexion of the membrane stretched across the opening without increasing the pressure to be applied to the membrane from below. Similarly, vacuum can be applied to the flexible membrane to cause the fluid to move in the flow channel in a reciprocating or oscillating fashion. Thus the fluid in the flow channel can be caused to move through the horizontal section of the flow channel by applying positive or negative pressure to a flexible membrane across the inlet channel opening on the bottom surface of the manifold body.

Pressure can be applied to the flexible membrane by a piston or similar solid component pushing the flexible membrane upward into the inlet (mechanical flexion), or a gas (pneumatic flexion) or liquid (hydraulic flexion) can be used to apply increased or decreased pressure (partial vacuum) to the flexible membrane to move fluid in the inlet channel. Regardless of how membrane flexion is induced, flexing the membrane across the opening of the inlet or outlet channel moves the fluid in the horizontal section of the flow channel, and can be used to provide a continuous reciprocating movement of fluid. Membrane flexing devices are known in the art for some applications in cell culture methods, such as the Flexcell® FX6000TM Tension system that uses pneumatic flexion produced by pressurized gas or vacuum or both, and can be adapted for use as the driving force to provide fluid motion in the devices and methods of the invention.

Suitable materials for the flexible membrane include silicone, natural rubber, latex, PDMS, or TPU. Optionally, a rigid plate can be added underneath the flexible membrane to hold the flexible membrane in place on the bottom of the manifold body (see support plate 18 in FIGS. 5A and 5B), although other methods of holding the membrane in place can be used, such as clamping the manifold body down onto a surface with the flexible membrane held between the surface and manifold body. Optionally, one or more O-rings (see 11a in FIG. 5B) can be interposed between the manifold body and the flexible membrane to enhance the seal. Either vacuum or pressure (e.g., pressurized air or nitrogen) can be applied to the underside of the flexible membrane, causing the membrane to flex where it contacts fluid in an opening in the lower surface of the manifold body, e.g., the opening of the inlet channel connected to the flow channel. Optionally, gas pressure alternating with vacuum can be applied to increase the range of motion achieved with a particular manifold body configuration and flexible membrane. This fluid driving design permits close control of the shear stress affecting the living cells, and can be maintained for a prolonged period of time (e.g., several days), as is required for many types of tests.

In a preferred embodiment, the manifold body consists of two main parts, an upper plate having holes through it and a lower plate having recesses in its upper surface to provide the flow channels. The holes are generally cylindrical and perpendicular to the flat upper surface of the upper plate, and extend all the way through the upper plate. The ‘ceiling’ of the flow channels in this embodiment is the bottom surface of the upper plate, so the holes through the upper plate align with a recess in the lower plate. The recesses in the top surface of the lower plate thus define the form of the flow channel. See FIG. 4B. This configuration makes it easy to machine the manifold body, and requires the mating surfaces of the two parts, i.e., the lower surface of the upper plate and the top surface of the lower plate, to both be substantially flat aside from the features described herein, and it requires the two plates to form a water-tight seal when pressed together.

Typically, the flow channels in these embodiments of the invention are approximately as wide as the diameter of the holes, so that when a well of a multi-well cell culture plate is pressed into the hole from above, the lower surface of the well aligns with the ceiling of the flow channel, and most or all of the area of cell growth on the bottom of the well extends across a corresponding flow channel, so that fluid flowing through the channel provides shear stress across most or all of the cell growth area. This alignment promotes smooth and consistent shear stress when fluid flows through the flow channel.

Fluid dynamics simulations were used to test various configurations for the flow channels within the manifold body of the fluidic devices, to determine what flow channel shape provided more uniform shear stress in the region of the flow channel that intersects with the hole extending downward from the top surface of the manifold body, i.e., the region where cells on the bottom of a well of a cell culture plate fitted to the manifold body would be in contact with the fluid moving within the flow channel. The testing was intended to optimize consistency of shear across the region where living cells would be in contact with the moving fluid, in configurations where the vertical depth of the flow channel is uniform along the entire horizontal section of the flow channel, which simplifies construction. In particular, flow channels with straight sides from end to end were compared to ones having a simple convex curvature from end to end (where the flow channel is wider between the inlet channel and outlet channel), and with flow channels having complex or compound curvature from end to end. See FIG. 7A, showing fluid dynamic simulation results. These simulations indicate that a smooth transition between a narrower width channel at the ends of a flow channel (where it typically connects to the inlet channel at one end and the outlet channel at its other end), and a wider flow channel width in the center of the horizontal section of the flow channel between inlet channel and outlet channel, provides the most uniform fluid shear stress across the bottom of the wells. According to the calculations, the most uniform shear stress across a cell-growing region in this configuration is achieved by having a flow channel that is narrower than the cell-growing region at the ends, i.e. where the inlet and outlet connect with the flow channel, and about the same width as the cell-growing region in each place where the flow channel passes underneath a well, i.e., the region where the hole to accommodate a well intersects with the flow channel, and using a smooth curving profile (concave arc) along the length of the side walls of the flow channel so that the flow channel gets wider in the intersection region than it is at the ends where the flow channel connects to the inlet channel and outlet channel. See FIG. 7B, showing a top view of the shape of two versions of the flow channel with smooth curvature. The version on the right in FIG. 7B corresponds to Trial 2 of the simulation, and gives a more uniform shear stress across the critical zone where the fluid in the flow channel is passing across the bottom of a well fitted into one of the holes in the top surface of the manifold body, which corresponds to the zone where living cells would be adhered to the well when the device is in operation. The white circle illustrates the bottom of a well of cell culture plate.

Thus, in some embodiments, the horizontal section of the flow channel is narrower at the ends where it connects to inlet channel and outlet channel, and wider in the center where it intersects the hole extending downward from the top surface of the manifold body. In a preferred embodiment, the change in width of the horizontal section of the flow channel provides a smooth arc from end to end of the flow channel, e.g. from about 20% to about 90% of the width of the hole at the ends of the horizontal section where the flow channel connects to the inlet and outlet channels, and increasing smoothly between the inlet and outlet channels to its widest width in the region where the horizontal section of the flow channel intersects each hole to a maximum width of about 80-120% of the width of the hole, in embodiments where the flow channel intersects just one hole. See FIG. 7B. In some embodiments, the width at the end of the horizontal section of the flow channel is about 50-80% of the width of the hole it intersects, and at the widest point where it intersects the hole, the flow channel width is about 80-110% of the width of the hole. Where the flow channel intersects more than one hole, its ends where it connects to the inlet channel and outlet channel are narrower than the holes, e.g. about 20% to about 80% of the width of the holes, and the width increases in the regions where it intersects each hole to a width of about 80-120% of the width of the hole; and the horizontal section of the flow channel between adjacent holes (or wells) narrows to approximately the same width as the width at the ends of the flow channel. This configuration provides relatively uniform shear force across the cell-growing region of the wells when they are in contact with moving fluid in the flow channel.

In one embodiment specially adapted for use with a 96-well plate sized like the Transwell® plates described above, the flow channel is about 50-80% of the width of the hole at the ends, and widens to about 85% to 98% of the width of the hole where the flow channel intersects the hole. This configuration causes the bottom of the well to rest partially on the upper edges of the flow channel rather than being narrower than the flow channel, because the bottom of the well is wider than the width of the flow channel at the point of intersection. Because the bottom of each well rests on the edges of the flow channel, the bottom of each well is precisely aligned with the ceiling of the flow channel, thereby reducing inconsistency in the flow properties at this critical region that could occur if the wells were not consistently positioned. This configuration provides uniform shear stress on cells adhered to the bottom of the wells of a cell culture plate as described herein. It also provides a structure for the two-piece version of the manifold body that can be readily machined from suitable or preferred materials, and provides a structurally stable device when assembled for use because it maximizes the amount of solid surface on the lower portion of the two-piece design that supports the upper portion of the two-piece manifold body. Designs using a uniform width from end to end of the flow channel that was wider than the cell-growing region did not provide such a solid, stable structures on the upper surface of the lower plate when sized to accommodate conventional 96-well format cell culture plates, because the design having narrower flow channels retains more surface to support the lower plate of the two-piece manifold body when the parts are pressed together. Thus the preferred configuration for the horizontal section of a flow channel in the 96-well device where a flow channel serves a single well is that shown in FIG. 7B, where the maximum width of the flow channel is less than the diameter of the bottom of a well (which is represented as a dark circle across the middle of the flow channel) of the multi-well cell culture plate (e.g., 85-95% of the width of the bottom of the well) to be used with that device, and the holes and flow channels are configured to position the well where it is centered on the midline of the flow channel, and thus the bottom of each well rests on the edges of the recess in the lower portion of the two-piece manifold body that form the flow channel for that well. The shape for a corresponding horizontal section of a flow channel serving three adjacent wells is shown in FIG. 7C, where the flow channel is wider as it passes beneath each well (represented by the dark circles), but the widest width of this flow channel is still less than the diameter of the well to be used with it so that the bottom of the well rests on the upper edges of the flow channel. This design is ideally suited for use with commercial 96-well cell culture plates such as Corning's Transwell® plates.

### Methods of Bonding Flexible Membrane to the Manifold

The surface to which the PDMS flexible sheet was to be bonded was first cleaned thoroughly with isopropyl alcohol. Next, using a coarse sand paper of grit 40-60, the autoclavable polyetherimide (ULTEM) manifold was repeatedly rubbed in a number 8 motion against a flat surface to create a fresh coarse surface with further smoothening of the surface using sand paper of grit 360. Once the sand paper residue was washed off using isopropyl alcohol, the dried surface was then chemically modified with either APTES ((3-Aminopropyl)triethoxysilane) or Poly[dimethylsiloxane-co-(3-aminopropyl)methylsiloxane] for 20 minutes at higher temperature of 50° C. to introduce silane groups on the ULTEM plastic. Once the chemical reaction was completed, the surface was washed with large quantities of isopropyl alcohol followed by one minute sonication in isopropyl alcohol. Next, both the silane modified ULTEM and PDMS flexible sheet were exposed to corona treatment for 1 minute. Immediately after the corona treatment, the PDMS flexible sheet and the ULTEM were chemically bonded with Kwik-sil™ silicone adhesive. The manifold with PDMS flexible sheet were placed at 80° C. for 12 hours followed by their use. The sheet was noted to be bound very firmly to the ULTEM plastic. Further, a round of autoclaving did not seem to have any impact on the bond strength between the ULTEM and PDMS. The reaction scheme below describes a likely mechanism of APTES reaction with ULTEM. Wu, et. al, Lab on a Chip 14, no. 9 (2014): 1564-1571. Other glues that can be used include Reltek A37 primer coating followed by bonding with Reltek B482 adhesive.

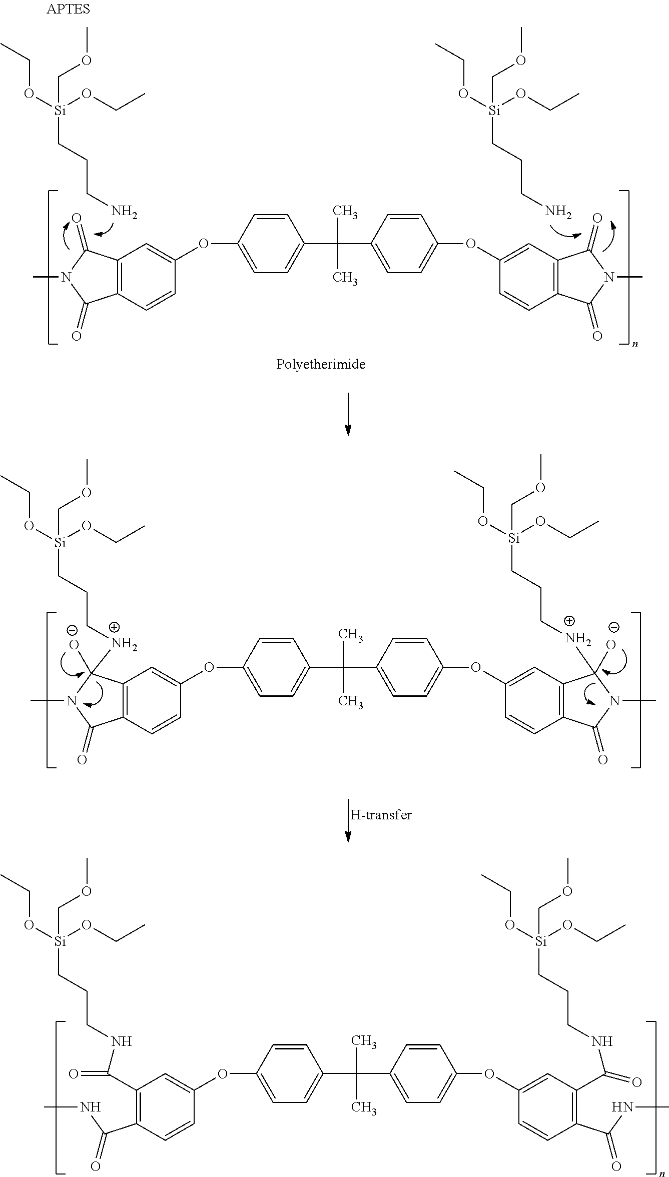

### Methods of Using the System

In another aspect, the present disclosure provides methods to use the fluidic devices described herein. The methods utilize a manifold body as described herein and a multi-well cell culture plate sized and configured to match and mate with the manifold body. It utilizes the manifold body to apply shear stress to cells on the bottom of a well of the cell culture plate, typically in a high-throughput format, e.g., using a cell culture plate having at least 16 wells, and typically at least 32 wells, and preferably at least 96 wells. For the methods, the flow channels contain enough fluid to at least fill the horizontal section of the flow channels, thus the fluid contacts the living cells adhered to the bottom of the wells. Preferably the fluid in the flow channels is moving across the bottoms of the wells to provide shear stress at least part of the time during operation. The movement can be a steady flow in one direction (e.g., from inlet channel to outlet channel) passing across the bottom of at least one well, or it can be a reciprocating motion where the fluid moves back-and-forth within the flow channel, so it generates shear stress on the living cells, but the direction of the flow and thus the shear stress reverses intermittently. This reciprocating flow has been found to simulate the phenotypic effects on shear-sensitive cells that are observed with unidirectional flow. Xu, et al, “GPR68 Senses Row and Is Essential for Vascular Physiology.” Cell 173, no. 3 (2018): 762-775.

Cells of a desired tissue or organ are grown on the underside of the porous membrane that forms the bottom of at least some of the wells of a multi-well cell culture plate such as a Transwell® plate. This can be done without using the fluidic device, or within the fluidic device but without fluid flow across the wells. Methods and conditions for establishing growth of cells adhering to the porous membrane are well known in the art, although normally the cells are grown inside the wells, e.g., on the upper surface of the membrane. Once cell growth is established and the cells form a stable matrix adhering to the membrane, the cell culture plate is removed from its initial medium, assuming this step is not done in the manifold body, and the cells are optionally rinsed before the multi-well cell culture plate is fitted to a manifold body of the invention. Fitting the culture plate to the manifold body requires inserting the wells into the corresponding holes in the manifold body and pressing the culture plate in place firmly enough to produce a liquid-tight seal between the wells of the plate and the sealing surfaces (e.g., O-rings) in the holes in the manifold body. A flexible PDMS or TPU membrane is then applied to the bottom of the manifold body, covering all of the holes in that surface that are fluidly linked to the inlets of the flow channels in the manifold body. The assembled device is then placed on top of a base that can apply force, either gas pressure or vacuum, to the flexible membrane where it covers each hole in the lower surface of the manifold body.

Once the device is assembled, a desired test sample is placed in each well, and a nutrient solution suitable for maintaining the living cells is added to the flow channels, for example via holes in the upper surface of the manifold body that are fluidly connected to the outlets connected to the flow channel. Once the two fluids are instilled and in contact with the cell matrix from above (the fluid in the wells) and below (the flow channel fluid), gas pressure or vacuum pressure is applied to the flexible membrane underneath the manifold body in a pulsatile manner, causing the fluid in the membrane to flex up and down, and causing fluid in flow channel to flow back and forth across the living cells. This applies a horizontal shear stress to the living cells across their lower surface. The frequency and magnitude of pressure changes, in combination with the elastic properties and thickness of the flexible membrane and the size and shape of the opening of the outlet flow channel that is in contact with the flexible membrane, as well as the dimensions of the flow channel can all be adjusted as needed to achieve a desired amount of shear stress on the living cells. In some embodiments, these parameters are adjusted so that the shear stress on the cells to be tested is sufficient to produce a desired phenotypic response in the cells.

If desired, a test substance can be added to either the fluid in the wells or the fluid in the flow channel (or both) to determine the effect of the test substance on the living cells. The properties of the cells, or changes in the composition of the fluid in the well and/or in the flow channel can be measured at suitable time intervals, or monitored continually, using methods well known in the art.

### Methods of Using the Device for a Multi-Organ Model System

In a further aspect, the present disclosure provides methods of using the devices and system of the invention to simulate two or more organs that may interact with each other. This can be accomplished with a single device, because the user can select which cell type to grow on each individual cell of the multi-well cell culture plate. Since each well can be isolated from the others when cells are being established on the outer surface of the well, the user can choose to have two, three or more different organs represented in one multi-well cell culture plate. A single plate can thus be used to explore multiple cell types of a given organ, or cell types from different organs. Moreover, a single plate can be used to model interactions between different cell types of an organ, or interactions between different organs.

In order to simulate a multi-organ system, the fluidic device can be configured with a single flow channel connecting at least two adjacent wells of the multi-well cell culture plate in the manifold body of the device. See, for example, FIG. 8, where one well represents a liver organ and the other well represents a kidney organ. Two (or more) different types of cells representing two (or more) different organs are then grown on two (or more) wells that share a single flow channel. The fluid in the flow channel is accessible to living cells (21) growing on both of the wells, and both types of cells interact with the same sample of fluid. Note that the cells are positioned at the ceiling (7) of the flow channel (3), so fluid flowing between the inlet channel (4) and outlet channel (5) flows across and in contact with the living cells. The opening (13) of inlet channel (4) is flared where it reaches the lower surface of the manifold body, and flexible membrane (17) stretching across the flared opening (13) can be deflected up or down to drive fluid motion within the flow channel. Thus, for example, metabolites from one cell type can reach the other cell type to interact with the other cell type, or to be further metabolized by the other cell type, when the motion of fluid in the flow channel moves fluid from the bottom of one well to the bottom of an adjacent well exposed to the same flow channel. The motion of the fluid moves effectors from one well to the other (and back), thus allowing cells on each well to come in contact with the effector and with any metabolite of the effector that is formed by either cell. Accordingly, the flow channel in such systems having two or more wells in contact with the fluid in a single flow channel simulates a circulatory system that allows movement of materials between two model organs, represented by two spatially separated groups of cells on two different wells of a Transwell® plate. It allows the two organs to effectively interact with one another and share via ‘circulation’ any effectors or signals that the first organ/tissue generates or is exposed to by the presence of the second organ/tissue.

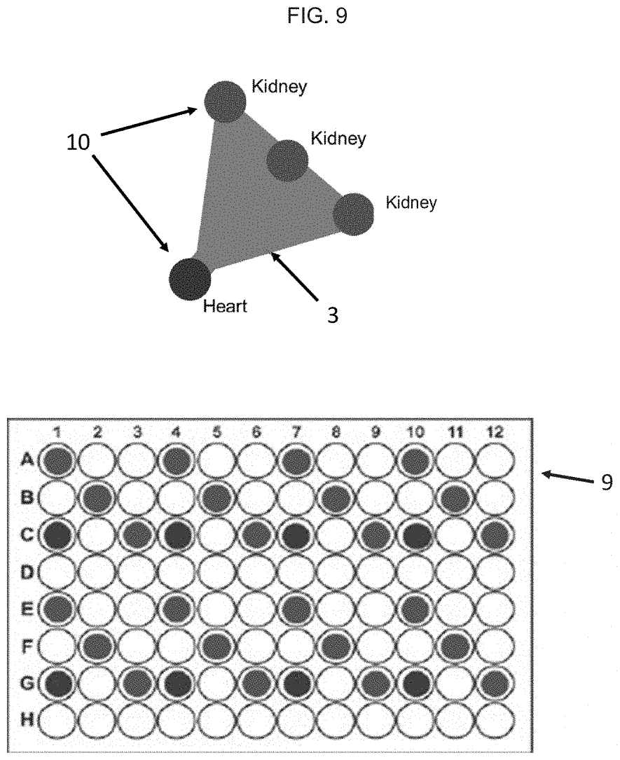

Optionally, a single flow channel can connect more than two wells, and can thus be used to connect three or more simulated organs. The wells can be sized differently, if desired, to represent the relative sizes of different organs. Alternatively, the system can be configured to have two, three, or four or more wells growing a first cell type, and a different number of wells (e.g., one) growing a second cell type: this permits the system to simulate an in vivo situation having more of the first tissue or organ than of the second, and allows the device to more accurately represent an in vivo multi-organ system, while having uniformly-sized wells and using commercially available multi-well plates. FIG. 9 depicts an arrangement of cell types on wells of a 96-well plate that exposes fluid in a flow channel to three times as many kidney cells as heart cells, illustrating one way the device can be used to simulate different proportions of cell populations or activities among interacting organs. Note that in the multi-well cell culture plate (9) for this embodiment, only the wells shown as dark circles would have living cells on the bottom surfaces, in a pattern that corresponds to the wells (1) indicated as Heart or Kidney, and that flow channel (3) broadens from the zone where it intersects the well having heart cells to a width spanning across three wells having kidney cells, thus allowing simulating a ratio of one heart cell to three kidney cells in the interaction between the living cells and the fluid in the flow channel.

### EXAMPLES

The following examples are provided to illustrate the invention, not to limit it.

### Example 1. Device Having a Two-Piece Manifold Body

A block of ULTEM or other suitable machinable plastic of the desired dimensions for each layer of the two-piece manifold body shown in FIGS. 4A and 4B was prepared, including small ears (19) around the perimeter that serve to receive screws for connecting the two pieces together or for attaching an added layer to the two-piece manifold body, or for detaching a multi-well cell culture plate that has been fitted to the manifold body. These small ears are depicted in FIG. 5 with screws connecting the separate portions of the manifold body. This manifold body is sized to match the horizontal dimensions of a chosen cell culture plate such as a commercial 96-well cell culture plate such as a Transwell® plate by Corning. Holes are bored through the top portion in an 8×12 array to match the position and sizes of the wells of the chosen cell culture plate. A groove is then machined within each hole around its perimeter, near the bottom of the top portion of the two-piece manifold body, to hold an O-ring that is sized to provide a liquid-tight seal between the walls of the hole in the upper portion and the outside of the wells of the chosen cell culture plate. See the groove 12 in FIG. 1. Note that in FIG. 1, the shading indicates the two separate portions of a two-piece manifold body. Additional smaller holes are then bored through the upper plate to provide outlet flow channels, positioned to connect to one end of the horizontal section of a flow channel that will be formed within the manifold body by the canals in the lower portion. See FIG. 1. O-rings are inserted into the grooves in each of the holes to receive wells. FIG. 2 depicts a single unit of a device, showing how O-ring (11a) seals around a well when it is inserted into the manifold body. FIG. 5A depicts the O-rings as an array of rings (11) positioned between the upper and lower portions (14 and 15, respectively) of the two-piece manifold body, though the O-rings may be positioned within the holes through the upper portion of the manifold body (14).

Recesses in the shape of shallow canals, typically-4 mm deep, are machined into the top surface of the lower portion of the two-piece manifold body (FIG. 4B), positioned to align with the holes in the upper portion for receiving wells of the chosen cell culture plate and to provide the pattern of flow channels desired. The flow channels are shaped according to the fluid dynamic simulation results described above (FIG. 7B, 7C): narrower at the ends and wider in the middle, with the width changing in a smooth arc from end to end. Holes are then drilled through the end of the flow channel opposite the end where the inlet channel is positioned, to provide the inlet channels of the device shown in FIG. 4B. Where these inlet channel holes emerge on the bottom surface of the lower portion of the two-piece manifold body, each hole is optionally enlarged or flared as shown in FIG. 6, to the extent needed to provide a desired degree of fluid movement. The enlarged, or flared, opening of the outlet channel provides a larger area for the flexible membrane to flex when pressure is applied, and thus increases the amount of fluid motion occurring in the flow channel for a given amount of pressure applied to the flexible membrane where it spans the opening of the outlet channel.

FIG. 4C depicts both upper and lower surfaces of the upper portion of a manifold body, and FIG. 4D depicts both upper and lower surfaces of the lower portion of a manifold body. Inside each hole through the upper portion of the two-piece manifold body, FIG. 4C shows the grooves that hold O-rings (11a) inside each of the holes (6) configured to accept wells of a Transwell® plate. On the bottom view of the lower portion of the manifold body, in FIG. 4D, are shown grooves (13b) configured to hold O-rings (11b) around each of the flared inlet openings (13a).

The upper and lower portions of the two-piece manifold body are then glued together with E6000 glue or B482 Reltek® adhesive. The glue holds the two portions securely together and ensures a liquid-tight seal between the two portions of the manifold body.

Device for Use with Membrane-Driven Fluidic Motion

A flexible membrane made of PDMS, approximately 1-2 mm thick, is applied to the bottom of the assembled two-piece manifold body described above. A support plate made of a rigid plastic or metal and having holes positioned to align with the openings of each of the outlet channels on the bottom of the two-piece manifold body is then applied to the underside of the flexible membrane to hold it in place. This support plate is held in place by screws that connect it to the upper or lower portion of the two-piece manifold body. This support plate (18) is depicted in FIG. 5.

A chosen cell culture plate, which can be a Transwell® plate from Corning, is fitted to the top of the manifold body, with each of the wells of the cell culture plate inserted into one of the holes through the upper portion of the two-piece manifold body. The cell culture plate is pressed firmly into the manifold body, so that each well forms a fluid-tight seal with the O-ring inside its corresponding hole. When preparing the assembly to perform testing on living cells, the chosen cell culture plate has living cells adhered to the permeable membrane forming the bottoms of at least some of the wells as described herein. Each well is partially filled with an aqueous medium compatible with the living cells adhered to the permeable membrane in order to protect and hydrate the cells.

Where appropriate for the particular experiment, a test substance can be added to the aqueous medium.

### Example 2

The assembly of Example 1 is placed atop a membrane flexion device configured to apply vacuum, gas pressure, or both to the underside of a flexible membrane. Optionally, the membrane flexion device has a platform sized for the manifold body to sit entirely on the platform, and the platform has vent openings sized and positioned to match the pattern of inlet openings on the lower surface of the manifold body. Each of the inlet openings in the manifold body aligns with one of the vent openings in the platform. The membrane-flexion device is adapted to deliver either vacuum or pressure or both via the vent openings, thus directing force to the flexible membrane on the bottom surface of the manifold body. The membrane flexion device can deliver either upward force via pressurized gas, or downward pressure via vacuum (reduced pressure) to the membrane (or both) at each of the vent openings. See FIG. 6.

A cell culture fluid is added to each of the flow channels via the outlet flow channel openings in the top surface of the manifold body. Enough cell culture fluid is added to fill the horizontal section of the flow channel and the inlet channel (which extends downward from the horizontal section of the flow channel): typically, enough fluid is added to also partially fill the outlet channel, which ensures that the horizontal section of the flow channel remains filled with the cell culture fluid during operation. Where appropriate for the particular experiment, a test substance can be added to the cell culture fluid. If needed, the assembly with fluid inside the flow channels can be vibrated or centrifuged to assist with removing any air bubbles inside the horizontal section of the flow channel or the inlet channel. Further, throughout operation, the entire device manifold can be tilted, e.g., the horizontal section of the flow channel can be kept at a 30 degree incline, with the outlet channel on the higher end, so that any air bubbles travel through the channels and exit to the top section of the plate.

Once the flow channels are filled to the extent desired, the assembly of Example 1 is securely mounted on the platform of the membrane flexion device, and the membrane flexion device is activated. The membrane flexion device is programmed to deliver force, i.e., pressurized gas or vacuum (or both alternately) to the flexible membrane via the vent openings. The flow channel, vent openings, flexible membrane, and inlet channel flare are sized and configured to cause the membrane to flex enough to drive fluid motion within the horizontal section of the fluid channel at a sufficient velocity to provide a suitable level of shear stress on the cells. The force is applied in a pulsatile manner, causing the membrane to flex up and down, and thus causing fluid to flow back and forth within the horizontal section of the flow channel, which exerts shear stress on the living cells, connects different wells together and moves the metabolites from one well to another. The pulsatile application of force is continued for the duration of an experiment, i.e., for at least 5 minutes, typically at least one to two hours, and frequently for at least 24 hours. A more detailed description of this process is provided in the following examples.

### Example 3

Freshly isolated primary mouse derived proximal tubule epithelial cells (PTECs) are grown to confluence in a collagen I coated T75 flask in ATCC media before the experiment. ATCC media—Renal Epithelial Cell Basal Medium (ATCC® PCS-400-030™) is mixed with Renal Epithelial Cell Growth Kit (ATCC® PCS-400-040™) containing—Triiodothyronine: 10 nM, rh EGF: 10 ng/mL, Hydrocortisone Hemisuccinate: 100 ng/mL, rh Insulin: 5 μg/mL, Epinephrine: 1 μM, L-Alanyl-L-Glutamine: 2.4 mM, Transferrin: 5 μg/ml Fetal bovine serum (FBS): 0.5% as per vendor's recommendations.

The surface of a 96-well Corning Transwell® insert plate is prepared a day before the cell culture. The underside of the Transwell® insert plate is coated with either a thin layer of collagen I gel, using Bovine Type I Collagen Solution, PureCol® in DMEM/F-12 Medium, 5 mg/ml (10 μL), or incubated in pH neutralized collagen type I solution from Corning for coating the Transwell® insert membrane at a concentration of 5 to 10 μg/cm² for 1 hour at 37° C. incubator to ensure coating. Once the gels are washed with 1×PBS, the Transwell® insert plates are placed in a 4° C. refrigerator until use. Following the proper collagen coating, freshly trypsinized PTECs are added to the collagen I coated Transwell® insert plate at a concentration of 200,000 cells/cm². The cells are added to the underside of the Transwell® insert plate and are allowed to adhere to the surface for 3 hours, then 3 hours later, more medium is added to allow cell adhesion and expansion over the next 21 hours.

The following day, the freshly autoclaved device is assembled together by positioning the components in alignment and tightening the appropriate screws around the perimeter of the manifold body (see FIG. 5). The device is then placed at a 30 degree incline, with the outlet channels at the high end of the flow channel. Next, 200 μl of fresh medium is carefully added to the channels to fill up the flow channel and the inlet channel without any introduction of air bubbles. Further, any air bubbles that might be generated are pushed to the vertical outlet section due to the incline. Next, the Transwell® insert plate with the cells is carefully removed and pressed into the manifold body of the device. The entire assembly is slightly rocked to remove air bubbles via the vertical outlet section.

Next, 30 μL of fresh medium is added via the outlet vertical section to fill up the vertical outlet channel and prepare the device for pneumatic flexion and introduction of appropriate shear. The entire device is briefly centrifuged to remove any remaining air bubbles at 120 r.c.f for 60 seconds. The device with the Transwell® insert plate is then placed securely onto the membrane flexion device to introduce regulated rhythmic suction to the flexible membrane of the device. Rhythmic suction-created fluid movement introduces a steady fluid shear stress of 0.2 dynes/cm². The medium is refreshed everyday via the outlet channel in the top surface of the manifold body. After 9 days of fluid shear, the system is disassembled by slowly turning the dislodging screws counter-clockwise around the manifold body. The cells on the Transwell® insert are then fixed and stained for detecting expression of markers such as ZO-1, acetylated tubulin, Na—K ATPase and aquaporin followed by their imaging.

### Example 4: Multi-Organ Kidney—Liver System to Establish a Multi-Organ Nephrotoxic Model of Acute Kidney Injury

Freshly isolated primary mouse derived proximal tubule epithelial cells (PTECs) are grown to confluence in a collagen I coated T75 flask in ATCC media before the experiment. ATCC media—Renal Epithelial Cell Basal Medium (ATCC® PCS-400-030™) is mixed with Renal Epithelial Cell Growth Kit (ATCC® PCS-400-040™) containing—Triiodothyronine: 10 nM, rh EGF: 10 ng/mL, Hydrocortisone Hemisuccinate: 100 ng/mL, rh Insulin: 5 μg/mL, Epinephrine: 1 μM, L-Alanyl-L-Glutamine: 2.4 mM, Transferrin: 5 μg/ml Fetal bovine serum (FBS): 0.5% as per vendor's recommendations.

The surface of a 96-well Corning Transwell® insert plate is prepared a day before the cell culture. The underside of the Transwell® insert plate is coated with either a thin layer of collagen I gel using Bovine Type I Collagen Solution, PureCol® in DMEM/F-12 Medium, 5 mg/ml (10 μl) or incubated in pH neutralized collagen type I solution from Corning for coating the Transwell® insert membrane at a concentration of 5 to 10 μg/cm² for 1 hour at 37° C. incubator to ensure coating. Once the gels are washed with 1×PBS, the Transwell® insert plates are placed in 4° C. refrigerator until use. Following the proper collagen coating, freshly trypsinized PTECs are added to the collagen I coated Transwell® insert plate at a concentration of 200,000 cells/cm². The cells are added to the underside of the Transwell® insert plate and are allowed to adhere to the surface for 3 hours, then 3 hours later, more medium is added to allow cell adhesion and expansion over the next 21 hours.

The following day, the freshly autoclaved manifold device components (a version of the manifold where every well gets its own fluidic connection) are assembled together by aligning the sections of the manifold body and tightening the appropriate screws around the perimeter (see FIG. 5). The device is then placed at a 30 degree incline, with the outlet channels at the high end of the flow channel. Next, 200 μl of fresh media is carefully added to the channels such that they fill up the flow channel and the inlet channel without any introduction of air bubbles. Further, any air bubbles that might be generated are pushed to the vertical outlet section due to the incline. Next, the Transwell® insert plate with the cells is carefully removed and pressed into the manifold body of the device. The entire assembly is slightly rocked to remove air bubbles via the vertical outlet section.

Next, 30 μl of fresh medium is added via the outlet vertical section to fill up the vertical outlet channel and prepare the device for pneumatic flexion and introduction of appropriate shear. The entire device is briefly centrifuged to remove any remaining air bubbles at 120 r.c.f for 60 seconds. The device with the Transwell® insert plate is then placed securely into the membrane flexion device to introduce regulated rhythmic suction to the flexible membrane of the device. Rhythmic suction-created fluid movement introduces a steady fluid shear stress of 0.2 dynes/cm². The medium in the flow channels is refreshed everyday via the outlet channel in the top surface of the manifold body. Chang, et al. “Human liver-kidney model elucidates the mechanisms of aristolochic acid nephrotoxicity.” JCI Insight 2, no. 22 (2017).

In the diagonally adjacent well to the proximal tubules, which lies ahead (upstream) of the proximal tubule well, freshly prepared hepatocyte single cells or 3D spheroids (7 days old) are sandwiched in thin layer of collagen I or matrigel either in the upper section of the Transwell® or under the Transwell® membrane. The hepatocyte spheroids are prepared as described below. Bell, et al. “Characterization of primary human hepatocyte spheroids as a model system for drug-induced liver injury, liver function and disease.” Scientific reports 6 (2016): 25187. Seven days prior to the multi-organ co-culture, 500-1000 primary hepatocytes (human or mouse, determined by the origin of proximal tubule cells) are plated in 384 well ultralow attachment Corning plates in 100 μl Williams E medium supplemented with 2 mM L-glutamine, 100 units/ml penicillin, 100 μg/ml streptomycin, 10 μg/ml insulin, 5.5 μg/ml transferrin, 6.7 ng/ml sodium selenite, 100 nM dexamethasone, and 10% FBS. The cells are centrifuged at 100 r.c.f for 5 minutes to trigger spheroid formation. The medium on the spheroids is replaced every day after day 3 until day 6. On day 6, the spheroids are collected using bravo tool and 50-100 spheroids are sandwiched either above or below the Transwell® membrane in a very thin layer of dilute collagen or matrigel (0.23 mg/ml). The hepatocyte spheroids are maintained in the fluidic manifold for 24 hours before connecting proximal tubule cell unit downstream. In case of plating 2D hepatocytes, the hepatocytes are plated upstream to proximal tubule cells 7 days in appropriate hepatocyte media prior to the Transwell® plate being moved to multi-organ connection. As mentioned before, both the PTECs and hepatocytes are initially cultured in manifold with fluidic connections to individual wells with their specialized media. After 7 days, they are moved to a connected system, where hepatocytes (sandwich cells or spheroids) are upstream of PTECs. 1:1 ratio of PTEC media and hepatocyte media are used to maintain the PTEC and hepatocyte co-culture. On day 7, nephrotoxic drug aristolochic acid which requires a hepatocyte metabolism step before inducing nephrotoxicity is added to the liver hepatocyte well at a concentration of 0, 10, 20 and 30 μM for 24 hours followed by live dead staining of the PTEC well to study hepatocyte induced nephrotoxicity of aristolochic acid.

### Example 5—Testing Well to Well Communication with Hoechst Nuclear Stain as a Small Molecule Surrogate

20,000 HCT116 cells were plated directly on the upper side of a Transwell® membrane (catalog number 3386 for Transwell® plate) in McCoy's 5A media such that the connected wells preceding them were either filled with McCoy's 5A media (50 μls) alone or filled with 25 μls of 2.5 mg/ml collagen gel with McCoy's 5A media (50 μls) above it.

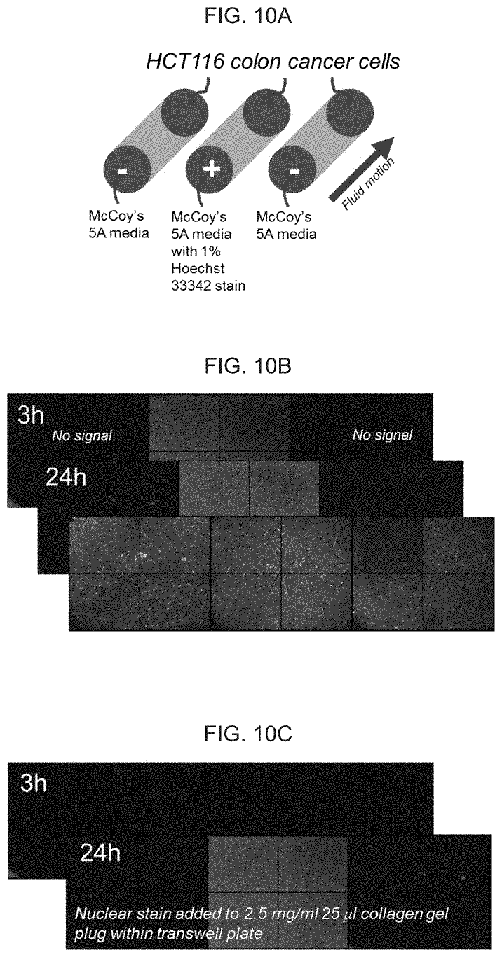

The high-throughput microfluidic device illustrated in FIG. 5A was cleaned, assembled and sterilized via autoclaving for 60 minutes. The wells of the autoclave-sterilized high-throughput microfluidic device with two wells connect for each test unit were filled with 300 μLs of media and centrifuged at 40 r.c.f for 4 minutes to fill all the channels. Transwell® plates with the HCT116 cells adhered on the upper side of the membrane were carefully lowered into the channels and pressed in to make seamless contact with the microfluidic channel. The set-up is illustrated in FIG. 10A, which shows three testing units, each consisting of a pair of connected wells, where the lower well in each test unit was charged with medium only, while the upper well in each test unit contains HCT116 cells on the upper surface, inside the well.

The first experiment with this setup was to demonstrate communication between the medium-only wells and the connected wells containing HCT116 cells. After 24 hours of culture, 50 μls of fresh media with 1:1000 (v/v) ratio of 10 mg/ml Hoechst nuclear stain was added to the lower well in the center pair, while both of the adjacent pairs of wells remain without the stain (FIG. 10A). The device was placed on compressed air system and the fluid was pumped via membrane flexion under a compressed air of 1.5 psi (amplitude), sinusoidal wave pattern with a frequency of 0.2. The cells were imaged after 3 hours and 24 hours to understand the diffusion characteristics of the nuclear stain from the connected wells to the wells with cells.

### Observations

FIG. 10B shows images of the cells in the upper wells of test units without collagen gel. Each row in FIG. 10B shows the three cell-containing wells, with each well divided into quadrants. The front row in FIG. 10B has a live/dead staining, just to demonstrate that the cells in each well were live after 24 hours of culture time, before the device was activated. The back row in FIG. 10B shows that cells were moderatly stained after 3 hours of operation, and the middle row shows that staining was even more extensive after 24 hours of operation. Thus FIG. 10B shows that the wells that did not have a collagen gel plug underwent rapid diffusion, mixing and distribution of the nuclear stain from the medium well to the cell-containing well. After 3 hours, almost no nuclear stain signal was observed in the adjacent wells (adjacent to the wells without the collagen plug), and only very slight nuclear stain appears in the adjacent wells after 24 hours of operation. Thus virtually no cross-contamination of nuclear stain to the wells adjacent to test units without nuclear stain was observed.

FIG. 10C shows similar images for the cell-containing wells in the collagen-containing system after 3 hours and 24 hours of operation. There was a significant delay in diffusion of the nuclear stain due to the collagen plug, as shown by the very weak staining at 3 hours, and no staining of the adjacent wells was observed in these wells.

After 24 hours, both the conditions (with and without collagen gel plug) showed strong nuclear staining in the cells in the well connected to the stain-containing well. Hoechst stain added to the collagen gel plug underwent slower diffusion due to the gel barrier, showing little or no staining of cells in connected wells in 3 hours, but showed ample staining after 24 hours. We again observed no leakage of the nuclear stain as noted by absence of any staining to cells in the wells surrounding the nuclear stain added sample, indicating no leakage of contents into the adjacent non-connected wells.

These results confirm the fluidic crosstalk between connected wells in the operating device, demonstrating exchange of small molecule constituents between connected wells with minimal leakage of the stain into adjacent non-connected wells.

### Example 6—Staurosporine Acute Toxicity on HCT116 Colon Cancer Cells (24 Hours)

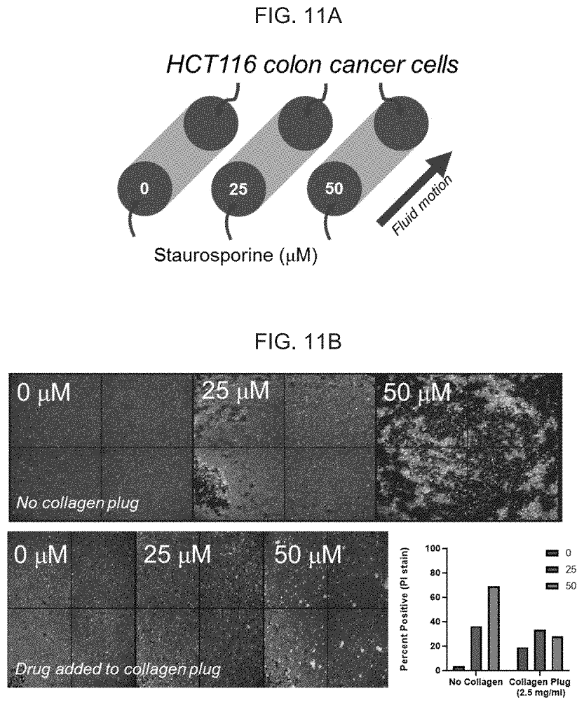

20,000 HCT116 cells were plated directly on the Transwell® membrane (catalog number 3386 for Transwell® plate) in McCoy's 5A media such that the connected wells preceding them were either filled with McCoy's 5A media (50 μls) alone or filled with 25 μls of 2.5 mg/ml collagen gel, with McCoy's 5A media (50 μls) above it. The arrangement is depicted in FIG. 11A, showing three pairs of connected wells (test units), where the lower well in each connected pair (test unit) has medium only, while the upper well in each test unit has HCT116 cells inside the well.

The high-throughput microfluidic device of FIG. 5B was cleaned, assembled and sterilized via autoclaving for 60 minutes. The wells of the autoclave sterilized high-throughput microfluidic device (two-well connects) were filled with 300 μLs of media and centrifuged at 40 r.c.f for 4 minutes to fill all the channels. Transwell® plates with the cells adhered on the upper side of the membrane (inside the wells) were carefully lowered into the channels and pressed in to make seamless contact with the microfluidic channel.

After 24 hours of culture, 50 μls of fresh media with different concentrations of staurosporine were added to different wells (see FIG. 11A). The device was placed on a compressed air system and the fluid was pumped via membrane flexion under a compressed air cycle of 1.5 psi (amplitude), sinusoidal wave pattern with a frequency of 0.2. The cells were imaged with live/dead staining after 24 hours to understand the diffusion characteristics of the drug and resulting cell death induced by staurosporine. The images of the cell-containing wells are shown in FIG. 11B, with the upper panel showing results in the test units without collagen gel, and the lower image showing staining results for the test units having collagen. Note that each image of the cells in a single well is divided into quadrants. The first image, without staurosporine, shows live cells. The second image in the top panel shows significant cell death, with much more cell death shown in the third image (the dark spaces represent areas devoid of live cells), corresponding to 50 uM staurosporine, Thus the collagen-free experiment shows dose-dependent delivery of staurosporine from the cell-free well to the cell-containing well in each test unit, as demonstrated by the extent of cell death.

The units with collagen gel plug acting as a diffusion barrier to the small molecule drug show that diffusion of staurosporine was significantly slowed by the collagen plug. In these images in the second panel in FIG. 11B, only a minor increase in cell death was observed in cell-containing wells connected to the well with higher concentration of staursporine, and cell death was substantially lower than in the test units without the collagen gel plug. The bar graph in FIG. 11B quantifies the effect of staurosporine in the collagen-fee test units 0 uM, 25 uM, and 50 uM staurosporine, and in the collagen-plugged test units at the same staurosporine concentrations. The results show very effective and dose-dependent transfer of staurosporine across the test units without collagen, and substantial but incomplete slowing of transfer of staurosporine due to the collagen plug.

These results confirm the fluidic crosstalk between connected wells in a test unit, exchange of small molecule constituents, and resulting functional consequence (cell death) in the adjacent connected well.

### Example 7—Hepatocyte—Cancer Cell Crosstalk

20,000 primary human hepatocytes (Lonza) (Hepatocyte plating medium for 4 hours, followed by hepatocyte maintenance medium with 0.3% (v/v) matrigel GFR for 20 hours) or HepG2 cells (to be transfected with CYP2A6 cDNA (as per vendor's recommendation)) were plated in one well of the two connected wells in their respective cell culture medium (catalog number 3386 for Transwell® plate). Prior to plating the primary human hepatocytes from Lonza, the Transwell® plate was coated with 0.1 mg/ml collagen (50 μls) (C3867 Sigma—collagen I rat tail solution) (solubilized in 0.1% acetic acid in 1×PBS (v/v)) for 1 hour at 37° C. (cell culture incubator) and washed with 1×PBS. The primary human hepatocytes were sandwiched in the human hepatocyte maintenance medium with 0.3% matrigel GFR (v/v) for the next 20 hours.

In the other connected well of each test unit, 20,000 HCT116 cells were plated directly on the Transwell® membrane in McCoy's 5A media. For HepG2 cells, the cells were transfected with CYP2A6 cDNA for 48 hours prior to any drug addition. On the day of drug addition, the HCT116 colorectal cancer cells were moved into hepatocyte media in order to prevent mixing of different mediums.

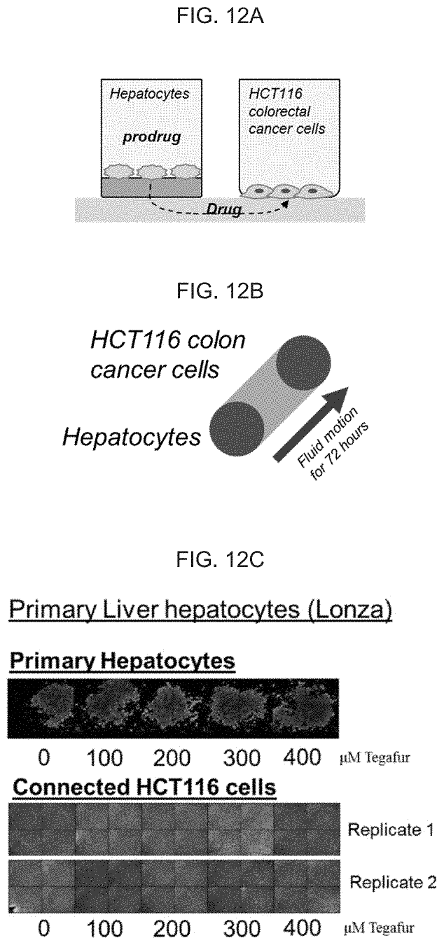

The high-throughput microfluidic device illustrated in FIG. 5B was cleaned, assembled and sterilized via autoclaving for 60 minutes. The wells of the autoclave-sterilized high-throughput microfluidic device (with two wells connected for each test unit) were filled with 300 μLs of media and centrifuged at 40 r.c.f for 4 minutes to fill all the flow channels. Transwell® plates with the cells adhered on the upper side of the membrane (inside the well) were carefully lowered into the channels and pressed in to make seamless contact with the microfluidic channel. FIG. 12A depicts how the wells are filled, and FIG. 12B illustrates a test unit of the system.

Next, different concentrations of prodrug Tegafur, a prodrug of 5FU (5-Fluorouracil), were added to the hepatocyte chamber, and fluidic flow was triggered for 72 hours. The device was placed on compressed air system and the fluid was pumped via membrane flexion under a compressed air of 1.5 psi (amplitude), sinusoidal wave pattern with a frequency of 0.2. Media was refreshed every 24 hours via the vertical channel with flared opening. The flared opening allowed easy physical and visual access into the constituents of the microfluidic channel in order to ensure proper filling. The cells were imaged with live/dead staining after 72 hours to understand the prodrug metabolism and resulting cell death in the HCT116 cells.

As seen in FIG. 12C, the first panel shows high viability of Hepatocytes was retained after drug treatment, with a slight dose dependent increase in the propidium iodide stain in the hepatocytes. However, the HCT116 eGFP cells in the connected wells showed significant dose dependent toxicity with increasing concentrations of Tegafur (second panel of FIG. 12C).

These results demonstrate the usability of the high-throughput multi-organ microfluidic device for complex biological experiments such as prodrug metabolization in one simulated organ (a first well) accompanied by cell death in a susceptible cell population in a second simulated organ (second well), where the drug liberated by cells in the first well resulted in death of cells in the second well.

### Example 8—Proximal Tubule Cell Culture on the Underside of the Transwell® Membrane Under Static Conditions and Shear Conditions

Human proximal tubule epithelial cells (PTECs) are grown to confluence in a collagen I coated T75 flask in ATCC media before the experiment. ATCC media—Renal Epithelial Cell Basal Medium (ATCC® PCS-400-030™) is mixed with Renal Epithelial Cell Growth Kit (ATCC® PCS-400-040™) containing—Triiodothyronine: 10 nM, rh EGF: 10 ng/mL, Hydrocortisone Hemisuccinate: 100 ng/mL, rh Insulin: 5 μg/mL, Epinephrine: 1 μM, L-Alanyl-L-Glutamine: 2.4 mM, Transferrin: 5 μg/ml Fetal bovine serum (FBS): 0.5% as per vendor's recommendations.

Transwell® insert membrane of 96 w corning plates were coated with 0.01% rat tail collagen (Sigma: C3867) (diluted in sterile cell culture grade water) by incubating the wells on a rocker for 6 hours at room temperature followed by their incubation overnight at 4° C. After rinsing the membranes with 1×PBS, 200,000 human PTECs/cm² (Primary Renal Proximal Tubule Epithelial Cells; Normal, Human (RPTEC) (ATCC® PCS-400-010™)) were added to the Transwell® membrane in 10 μL media and allowed to adhere to the membrane for 4 hours. The cells were then incubated with 200 μLs of media for 48 hours before the static culture.

For the fluidic shear experiments, the human PTECs were grown on the underside of the membrane for 6 days before transferring to the high throughput microfluidic device. A shear of 0.1-0.2 dynes/cm² was provided for a time period of 12 hours via rhythmic flexion of the flexible membrane of the device, followed by testing the PTEC cells for their viability.

Prior to the static or fluidic experiments, the high-throughput microfluidic device was cleaned, assembled and sterilized via autoclaving for 60 minutes. The wells of the autoclave sterilized high-throughput microfluidic device (two-well connects) were filled with 300 μLs of media and centrifuged at 40 r.c.f for 4 minutes to fill all the channels. Transwell® plates with the cells adhered to the underside of the membrane were carefully lowered into the channels and pressed in to make seamless contact with the microfluidic channel for 48 hours (static conditions) or 12 hours (fluidic shear conditions) followed by staining with calcein AM to qualitatively estimate the viability. Cells grown on the underside of a similarly prepared Transwell® membrane that wasn't inserted into the high throughput microfluidic device were used as a control for the experiment.

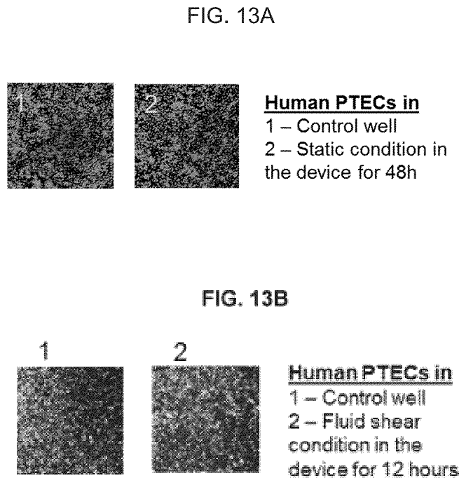

Cells cultured on the underside of the Transwell® membrane and transferred into the high throughput microfluidic device showed similar viability as compared to the control well that was not transferred (FIG. 13A). Cells exposed to fluid shear for 12 hours also showed similar viability to cells on the control membrane (FIG. 13B). This demonstrates that the microfluidic device can be used to expose healthy, live cells to shear flow conditions for testing as described herein.

What is claimed is:

1. A fluidic device comprising:
a manifold body comprising a substantially flat horizontal top surface, two or more separated flow channels, each of said flow channels having a horizontal section with a horizontal width, wherein each flow channel is connected to an inlet channel and an outlet channel, wherein the inlet channel and the outlet channel extend from the flow channel to the exterior of the manifold body;
the manifold body further comprising two or more holes extending downward from the top surface into the manifold body, wherein each of said holes intersects the horizontal section of one flow channel,
wherein each flow channel is configured so that fluid moving from the inlet channel through the horizontal section of the flow channel toward the outlet channel must pass through the portion of the flow channel that intersects at least one of the holes extending downward from the top surface of the manifold body; and wherein, optionally, the width of each hole where it intersects the flow channel is greater than the horizontal width of the flow channel at the point where the hole and flow channel intersect by a small amount.

2. The fluidic device of claim 1, wherein the holes that extend downward from the top surface into the manifold body are circular in horizontal cross section.

3. The fluidic device of any one of the preceding claims, wherein the two or more holes that extend downward from the top surface into the manifold body are sized and positioned to receive the wells of a multi-well cell culture plate.

4. The fluidic device of claim 3, which further comprises a sealing surface associated with each of said holes, wherein said sealing surface is configured to form a liquid-tight seal between a surface of the manifold body and an outer surface of each well of a multi-well cell culture plate when the multi-well cell culture plate is fitted to the top surface of the manifold body with the wells of the multi-well cell culture plate inserted into the holes that extend downward from the top surface into the manifold body.

5. The fluidic device of claim 4, wherein the sealing surface is a sleeve or an O-ring positioned inside each of the holes that extend downward from the top surface into the manifold body.

6. The fluidic device of claim 3, wherein an inlet channel or outlet channel for each flow channel extends upward from one end of the flow channel to open at the top surface of the manifold body.

7. The fluidic device of claim 3, wherein the holes that extend downward from the top surface into the manifold body are uniform in size and each hole is shaped as a right cylinder having a vertical central axis that is aligned with the midline of the horizontal section of the flow channel the hole intersects.

8. The fluidic device of claim 7, wherein the diameter of each of the holes is greater than or equal to the horizontal width of the flow channel where the hole intersects the flow channel so that the outer surface of the wells that are to be inserted into the holes when a multi-well cell culture plate is fitted to the top surface of the manifold body will extend beyond the flow channel and the bottom edge of the wells extends slightly over the top of the edges that define the flow channel.

9. The fluidic device of claim 7, wherein at least two of the holes that extend downward into the manifold body intersect with a single flow channel.

10. The fluidic device of claim 3, having a multi-well cell culture plate fitted to the upper surface of the manifold body so that each well of the multi-well cell culture plate is inserted into one of the holes in the top surface of the manifold body;
wherein at least one flow channel is configured to provide substantially uniform shear stress across the bottom surface of a well of the multi-well cell culture plate that is fitted to the top surface of the manifold body.

11. The fluidic device of claim 1, wherein the manifold body is a two-piece manifold body, which comprises an upper portion and a lower portion, wherein the upper portion has a substantially flat lower surface that is parallel to the horizontal top surface of the manifold body; and the lower portion has a substantially flat upper surface having canals formed therein, wherein the lower surface of the upper portion forms a ceiling over the canals when the upper portion and lower portion are assembled by being pressed together, and optionally having a gasket sandwiched between the upper portion and lower portion to provide a fluid-tight seal between the two portions, and optionally having the upper portion and lower portion glued together, to form an assembled manifold body, so that the canals in the upper surface of the lower portion taken together with the lower surface of the upper portion form the flow channels in the assembled manifold body.

12. The fluidic device of claim 1, further comprising a flexible membrane applied to a surface of the manifold body covering the openings of the outlet channels, wherein the flexible membrane forms a fluid-tight seal with the manifold body across each of the openings of the outlet channels, or wherein the manifold body comprises a groove around at least one of the outlet channel openings in the bottom surface of the manifold body, wherein each said groove is configured to hold an O-ring that is positioned to form a seal between the manifold body and the surface below it when the fluidic device is assembled, such as the flexible membrane when the manifold body is placed on a flexible membrane.

13. The fluidic device of claim 12, further comprising living cells adhered to a porous membrane that forms the bottom of a well of the multi-well cell culture plate.

14. A system for exposing living cells to fluid shear stress, which comprises a fluidic device according to claim 1; a multi-well cell culture plate configured for use with the fluidic device; and means to cause fluid inside the horizontal section of the flow channel of the fluidic device to move.

15. The system of claim 14, wherein living cells are adhered to the underside of the porous membrane of the wells of the multi-well cell culture plate.

16. A method to apply shear stress to a living cell, which comprises adhering living cells to the bottom surface of a well of a multi-well cell culture plate, and fitting the multi-well cell culture plate to the top surface of the fluidic device of claim 1.

17. A device to expose living cells to fluid shear stress, wherein the device comprises:
a plurality of wells having generally vertical walls and a generally horizontal floor, wherein at least a portion of the floor is a permeable membrane;
at least one flow channel positioned below the wells so that the permeable membrane portion of the floor of each well separates the well from one of the at least one flow channels;
an inlet that connects the flow channel to the exterior of the device, and an outlet that connects the flow channel to the exterior of the device, wherein a fluid path leading from the inlet, through the flow channel to the outlet passes beneath the permeable membrane portion of the floor of at least one well.

18. The device of claim 17, wherein living cells are adhered to the underside of the permeable membrane forming the floor of at least one of the wells.

19. The device of claim 17, which comprises at least two separate flow channels, wherein each of said flow channels is separated by the permeable membrane portion of the floor of a well from one well, or two wells, or three wells.

20. A method to use the device of claim 12, which comprises placing living cells on a permeable membrane of the device, and contacting the living cells with fluid moving through a flow channel within the device.
